SEQUENCE LISTING (30 sequences)

SEQ ID NO: 1 - DNA, 2401 bp, Arabidopsis thaliana
tagatcaagttttacaactttttttacgtacttaaaagattgataaaattgtacttgttgttgttgaccattgaaacatttccctttattcactaaaactagcttttgcaacctttttattcaactgtattggtttgttgtatgtttttattattcaactctatttaattattcatgtagcttaacatatcaataataactaacatgccataaattccgaagaagaaaaattgttataaaagaactatatgcacctaacaaaaaaccaacaaaaccatagtattttctttttgggttacataaaaatatctttgtatgaagaagaagaaagaatcattacacagctttcactcacaagattacaagtttgcaaattgcatataaggaaactcccaaaaaatatcatcaaagatcttttcagaacacaaaaaaaaaaaggtaaaagtatatctcttttgccatagttagactcaaaaatacgacatcgtttacctcacacgtgcacactcaccgacttacagaagaacccacacgtacgttgagggtactttagtcatttagcatcgaacgaaattgtctgagcggagaagaggagtttccgttatccctttatatattatctctctcacctttcttctctttttctttgatttttattaaatcaacaaaaatagaaaaaaaaacataaaaataaaaataaaaaatcttcacgtttcttctctctctctctctctctctctcgagccaccaaatctgaattagggtttttgagaatattcatcttttgatttcaaattcttcacccactgtgtaatttcactcgtcaggattcatctgcggaatcatgattacagattcgatcaccaacgcttctgctacttcagctccgagagattccggaaagaagaagagggtaacgttatcctctttgataaatctcattcctttctctgaaattgattcaaagttttgatttttaatggttttgttattgcattacgttttgcagaacaataagtcggctaagatgaagcagaacaagcttggtctccgtcgtgagcaatggctttctcaaggttaaaacttcatctttctctcctttaatgtttcaattctgcctgattcgtttggttagatttggttgtctatgtattgattttgggtttttgattttgatattagttgcggtgagcaataaggaagttaaagaggagaggagtgttaatcgtagtcaaaagcctcatcatgagagttcagataaggtgcgtagagaagaggataacaatggtgggaataatcttcttcatcatgagagttttatggagtcaccttcaaatagctctgttggtggtacatattcgagcactaacttcagtgggagaagtagcaggagtagtagtagcagcagtggcttttgctctggtaatataacagaagaggaaaatgtagacgatgatgatgatgggtgtgtggatgattgggaagctgttgctgatgcgttagcggctgaggaagagattgagaaaaagagtcgtcctcttgagtctgtgaaagagcaagtgagtgttggacaatcagcttctaatgtgtgtgattcgtcgattagtgatgcatcagatgttgtgggtgttgaagatccaaagcaggaatgcttgagagtgtcatcaaggaagcagactagtaatagagcttggaggctagatgatgaccttcgcccacaggggttacctaatttggcgaagcagcttagttttccggagttagacaagcgttttagctctgtggcgattccgtcttcatgtcccatatgctacgaagacttggacttgacggattcgaatttcctcccctgtccttgtggatttcggctctgtctgttctgccacaagaccatttgcgatggagatgggcgttgtccaggctgcaggaaaccctatgaacggaatatggtcaaggctgagactagtattcaaggtggtggtctaacaattcggttggctcgttcgtctagcatgttttgcaagttttaaaaggagaggtgcggttttctcaaccatgttgtcttttggaactcgagaacttaagctctgttttctatgtcatctatggttctaagtctgaaacactgtggtgatgatgtagaatgtgatgtgtgaatacataaaaggtggtacagaaaatgattcaaatacatttagatagtttcaataatgaatgctatgttctcttttctaattccatatgtttggtctgcatttattccttgtcaaacattattgaaggtttaagagttattttgttgctatggtgaatcctcttgacaagttactcatgaaccaaagcttgttttttagaatcaccattcaccagagatcaactctcattacttcaaattctttta

SEQ ID NO: 2 - DNA, 1863 bp, Arabidopsis thaliana
atggcattgtctctgctccggaagaacaaacatggattcgatatagtaatcagtgatgttcatatgcctgacatggacggtttcaagcttcttgagcatgttggtctagagatggacttacctgttatcatgatgtctgcggatgattcaaagagtgtggttctaaagggagtaacgcacggtgcggttgattaccttatcaagcctgtacgtatggaggcacttaagaacatatggcagcatgtagttaggaagaggagaagtgaatggagtgtaccggaacattctgggagcattgaggagactggcgagagacagcagcagcaacatagaggaggtggtggtggtgcagctgtttctggtggagaggatgcggtggatgataactcatcctcggttaacgaaggtaacaattggaggagcagttcacggaagaggaaagacgaggaaggagaagagcaaggagatgataaggacgaagatgcgtcgaatttgaagaaaccgcgtgtcgtctggtctgttgaattgcatcagcagtttgttgctgctgttaatcagctcggcgttgagaaggcggttcctaaaaagatcttagagctgatgaatgttcctggtctaacccgagaaaacgtagcaagtcacctccagaaataccggatatatctaagacggcttggaggggtatcgcagcaccaaggcaatcttaacaactcgtttatgacgggtcaggatgcgagcttcggacctctttcgacattgaatgggtttgatcttcaagcactagccgtcacaggtcagttacctgcacagagtcttgcacagcttcaagccgctggtttaggccggcctgcgatggtctctaagtcaggtttgccggtttcctccattgtggatgagagaagcatcttcagctttgacaacacgaaaacaagatttggagaagggcttgggcatcacgggcaacaaccccaacagcaaccacagatgaacttacttcacggtgtccccacgggtttacaacagcagcttcctatgggtaatcgaatgagtattcaacaacagattgctgctgttcgagctggaaatagtgttcaaaacaacggaatgctgatgcctctagcgggtcagcagtctttgcctcggggaccaccgcctatgctaacctcttcgcaatcatccatcaggcagccgatgttatcaaaccgcatttccgagagaagtggtttctctggaaggaacaatatccccgagagcagcagagtgttaccgacaagttacactaatctcacaacacaacactcatcaagctcgatgccttataacaacttccaaccagaacttcccgtgaacagtttcccgctggcaagtgcaccagggatatcagtaccggttcggaaagccacttcttaccaggaagaggttaacagctccgaagcgggtttcactacgccgagctacgacatgttcaccaccagacagaatgattgggatctgaggaatattggaatagcctttgactcacatcaggactcagaatccgctgcgttttccgcttcagaagcctactcttcttcgtccatgtcaagacacaacacgacagttgcagccaccgagcatggccgaaaccaccagcagccaccatcgggaatggtacagcaccatcaggtttatgcagacggaaacggtggttcagtgagggtgaaatcagagagagtggctacggatacagcaacaatggcgtttcacgagcagtatagtaatcaagaagatcttatgagcgcacttcttaagcaggaagggattgcaccggttgatggcgaattcgactttgacgcatactccatcgataacattccggtttga

SEQ ID NO: 3 - DNA, 1935 bp, Arabidopsis thaliana
atggtaaatccgggtcacggaagaggacccgattcgggtactgctgctggtgggtcaaactccgacccgtttcctgcgaatcttcgagttcttgtcgttgatgatgatccaacttgtctcatgatcttagagaggatgcttatgacttgtctctacagagagcagagagcgcattgtctctgcttcggaagaacaaagaatggttttgatattgtcattagtgatgttcatatgcctgacatggatggtttcaagctccttgaacacgttggtttagagatggatttacctgttatcaatctgaatgttttgaaacctttggttatagtgatgtctgcggatgattcgaagagcgttgtgttgaaaggagtgactcacggtgcagttgattacctcatcaaaccggtacgtattgaggctttgaagaatatatggcaacatgtggtgcggaagaagcgtaacgagtggaatgtttctgaacattctggaggaagtattgaagatactggcggtgacagggacaggcagcagcagcatagggaggatgctgataacaactcgtcttcagttaatgaagggaacgggaggagctcgaggaagcggaaggaagaggaagtagatgatcaaggggatgataaggaagactcatcgagtttaaagaaaccacgcgtggtttggtctgttgaattgcatcagcagtttgttgctgctgtgaatcagctaggcgttgacagtgagttaaaaacttgcttgcttatgcatttgtgtgtgtcgattggtaacattgtggaattccagaagtatcggatatatctgagacggcttggaggagtatcgcaacaccaaggaaatatgaaccattcgtttatgactggtcaagatcagagttttggacctctttcttcgttgaatggatttgatcttcaatctttagctgttactggtcagctccctcctcagagccttgcacagcttcaagcagctggtcttggccggcctacactcgctaaaccagggatgtcggtttctccccttgtagatcagagaagcatcttcaactttgaaaacccaaaaataagatttggagacggacatggtcagacgatgaacaatggaaatttgcttcatggtgtcccaacgggtagtcacatgcgtctgcgtcctggacagaatgttcagagcagcggaatgatgttgccagtagcagaccagctacctcgaggaggaccatcgatgctaccatccctcgggcaacagccgatattgtcaagcagcgtttcaagaagaagcgatctcactggtgcgctggcggttagaaacagtatccccgagaccaacagcagagtgttaccaactactcactcggtcttcaataacttccccgcggatctacctcgcagcagcttcccgttggcaagtgccccagggatttcagttccagtatcagtttcttaccaagaagaggtcaacagctcggatgcaaaaggaggttcatcagctgctactgctggatttggtaacccaagctacgacatatttaacgattttccgcagcaccaacagcacaacaagaacatcagcaataaactaaacgattgggatctgcggaatatgggattggtcttcagttccaatcaggacgcagcaactgcaaccgcaaccgcagcattttccacttcggaagcatactcttcgtcttctacgcagagaaaaagacgggaaacggacgcaacagttgtgggtgagcatgggcagaacctgcagtcaccgagccggaatctgtatcatctgaaccacgtttttatggacggtggttcagtcagagtgaagtcagaaagagtggcggagacagtgacttgtcctccagcaaatacattgtttcacgagcagtataatcaagaagatctgatgagcgcatttctcaaacaggtttga

SEQ ID NO: 4 - DNA, 599 bp, Artificial Sequence
  derived from Arabidopsis thaliana
gctttgaagaatatatggcaacatgtggtgcggaagaagcgtaacgagtggaatgtttctgaacattctggaggaagtattgaagatactggcggtgacagggacaggcagcagcagcatagggaggatgctgataacaactcgtcttcagttaatgaagggaacgggaggagctcgaggaagcggaaggaagaggaagtagatgatcaaggggatgataaggaagactcatcgagtttaaagaaaccacgcgtggtttggtctgttgaattgcatcagcagtttgttgctgctgtgaatcagctaggcgttgacagtgagttaaaaacttgcttgcttatgcatttgtgtgtgtcgatgtttgttttcttgtctaatctttaccgtattggggcattcaatgcagaagctgttcctaagaagatcttagagatgatgaatgtacccgggctaacgcgagaaaacgtagccagtcacctccaggtatatatgtgatcgaatagtattacattttttcattgttgatgtgttctaacagtggtaacattgtggaattccagaagtatcggatatatctgagacggcttggaggagtatcg

SEQ ID NO: 5 - DNA, 780 bp, Arabidopsis thaliana
atggccagagacggtggtgtttcttgtttacgaaggtcggagatgatgagcgtcggtggtatcggaggaattgaatctgcgccgttggatttagatgaagttcatgtcttagccgttgatgacagtctcgttgatcgtattgtcatcgagagattgcttcgtattacttcctgcaaagttacggcggtagatagtggatggcgtgctctggaatttctagggttagataatgagaaagcttctgctgaattcgatagattgaaagttgatttgatcatcactgattactgtatgcctggaatgactggttatgagcttctcaagaagattaaggaatcgtccaatttcagagaagttccggttgtaatcatgtcgtcggagaatgtattgaccagaatcgacagatgccttgaggaaggtgctcaagatttcttattgaaaccggtgaaactcgccgacgtgaaacgtctgagaagtcatttaactaaagacgttaaactttccaacggaaacaaacggaagcttccggaagattctagttccgttaactcttcgcttcctccaccgtcacctccgttgactatctcgcctgaatcgtcgccgccgttgactgtttcgactgaatcgtcggattcgtctccgccgttatctccggtggagatcttttccacctcgccactttcatctccgatagacgatgaagatgatgacgtgttgacgtcgtcgtcggaggaatcgccgattcgacggcagaagatgaggagtcccggattagattag

SEQ ID NO: 6 - DNA, 891 bp, Arabidopsis thaliana
gttgattctctctatctctctcacgagtcacgatcctactcttcttgatatggctgaggttttgcgtcccgagatgttagatatctctaacgacacttcttcattagcatcaccgaaacttcttcatgttcttgctgttgatgatagtatggttgatcggaagttcatcgagcggttactcagagtctcatcttgtaaagttactgttgtcgatagtgcgacaagagctttacaatatcttggattagatggagagaataatagttcggttggatttgaggatctgaagattaatttgataatgacggattactctatgcctgggatgactggatatgaactactgaagaagatcaaagaatcatcagctttcagagaaataccagttgtgattatgtcttcagagaacatcttgcctcgtatcgatagatgtcttgaagaaggagctgaagatttcttattgaagcctgtgaaattagctgatgtgaagagattaagagattctttaatgaaagctgaggaaagagcttttaaggatattatgcacaagagagagcttgaagctaatgatatctactcgcagctaaaacgcgcaaagatctgagtttatcagattccacgtacgatatgttctttaaaagctcaaagattcacacacatgcatgtcctgattctttcggcttacaatttttgagacattacggcttcttttgctgatagaaccaagactgatcaagagtgacaaagatgggtttttataagatagaaatgtaaattgtagacttggattcattagagaggagaagagatacattttttttttctttacatgagagatatgtaaaatggtggtgtgttattttggaagttaacatatgaatttcttttgtt

SEQ ID NO: 7 - DNA, 942 bp, Arabidopsis thaliana
atcctccaacattcgttgatcaatggctgaagttatgctaccgaggaagatggagattctcaaccattcttcaaagtttggatcaccggatcctcttcatgttcttgccgtcgacgacagtcacgttgatcgtaaattcatcgagcgtttgctcagagtatcttcttgcaaagttactgttgttgatagtgcaacaagagctctccaataccttggtttggatgttgaggaaaaatcagtcggttttgaggatttgaaggttaatttaattatgactgattactcaatgcccggtatgactggatatgaactcttgaagaagatcaaagaatcctcagcttttagagaagtaccggtggtaattatgtcctccgagaacattttgcctcgtattgatagatgtcttgaagaaggggctgaagatttcttactgaagccggtaaaactctcggatgtaaaaagattaagagattctctaatgaaggttgaagatttgtctttcacaaagagtattcagaagagagagctagaaacagagaatgtctaccctgttcactcgcagctcaaacgcgcaaagatctgagctctccgatgcaaattccgtgactggatcttagaaaaagccttttcatatacaggaagcttcaagactcaatgcacttgagctacactgatgattctcgccagcttaatgacctatacaattttccaacccctatggcctttttcgctgattgaattcagtgggtttattcatatagtagtaatgtaaatggtatagagatgagcaaaagctatattatatattatattcattagaggggtcatagcttttttccttacaagagatttgtaagctggcttaatagaagttgttaagaagcctcactttatgtgaataaatgattttgttagctaatcataaacaatcagaagtttcct

SEQ ID NO: 8 - DNA, 740 bp, Arabidopsis thaliana
caatctgctctcttttttattctgagtttgacaatggcggttggtgaggtcatgaggatggagattcccgccggtggagatttgactgttactactccggagttgcatgttcttgccgtcgatgatagtattgtggatcgtaaagtcatcgagaggttgcttagaatctcttcttgtaaagtgacgactgtagagagtggaactagggctttgcagtatcttggcttagatggaggcaaaggggcttctaatcttaaggatttgaaggtgaatttgatagtgacggattactcaatgccaggactttcaggatatgatctccttaaaaagattaaggaatcttcagcattcagagaagtaccagtagtgattatgtcatctgagaacatcttacctcgtatacaagaatgtctcaaagaaggagcagaggaattcttgttgaaaccggtgaagctagcagatgtaaagcgaatcaaacaacttataatgaggaatgaagctgaggaatgcaaaatcttaagccattctaacaagagaaagcttcaagaagacagtgatacatcatcatcaagtcatgatgatacttctatcaaggactcttcatgttcaaaacgaatgaaatcagaatctgaaaaccttttttctctactttgaattatatgtatagaggccaagaccttagcaaaaccattctgttaatattcctcaagaaagacaatgtttgagagagtcatcacact

SEQ ID NO: 9 - DNA, 1659 bp, Arabidopsis thaliana
atgactatggagcaagaaattgaagtcttggaccagtttccggttgggatgagagttcttgctgttgacgatgaccagacttgtctccgtattctccagactttgcttcagcgctgccaatatcacgttacaacaacgaatcaggcacagaccgcattggagttgttgagggagaacaagaataagtttgatcttgttattagcgatgtcgacatgccagacatggatggtttcaagctgcttgagcttgttggtcttgaaatggacttacctgtcataatgttatctgcgcatagcgatccaaagtatgtgatgaaaggagtcaagcacggtgcctgtgattatctgcttaaaccggttcgaattgaggagcttaagaacatatggcaacatgttgtgagaaagagcaaacttaagaagaataagagcaatgtgagtaatggttcaggaaactgtgataaagcaaacagaaaacgtaaagaacagtatgaagaggaggaagaggaagaaagagggaatgataatgatgatccaacggcgcagaagaagcctcgtgttctttggacgcatgagctgcacaataaattcctagcagctgttgatcatttaggcgttgagagagctgttccaaaaaagattctagatctgatgaatgttgacaaactcactagagagaatgttgcaagccaccttcagaaattccgcgttgctctgaagaaggtgtctgatgacgccattcaacaagctaacagggcggctattgactcacattttatgcaaatgaattctcagaaaggacttggtggcttctaccaccaccaccgcggaatacctgttggatccggtcagttccatggtggaaccacaatgatgaggcattactcttcaaataggaatcttggtcgtctgaattcccttggagcaggaatgttccaaccagtctcatcatcgtttcctcgtaaccataatgatggaggaaacatacttcagggtttgccgctagaagagcttcagatcaacaacaacatcaacagggcttttccaagctttacttcacaacaaaactctccaatggtagctcccagtaatctgttacttctcgagggtaacccgcagtcatcatctttaccctcaaacccgggtttttctcctcatttcgagatcagcaagcgtctagaacattggtcaaacgctgcattgtcaaccaacattccacagagtgatgttcattcaaaacctgacaccttggaatggaatgcgttctgcgactcagctagtccgctagtaaacccaaacctggatacaaatccggcatctctctgcagaaacacgggttttggatccacaaatgctgcacaaacagacttcttttatccattacagatgaatcagcagcctgcaaacaactcaggtccagtgacagaagctcaactgtttagaagtagcaatccaaacgaaggtttactcatgggacaacagaagcttcagagtggtttgatggcttctgatgctggttccttagatgatatagtcaattccttaatgacacaggaacagagccaatctgatttctcggaaggtgattgggatttggatggtttagctcactcggaacatgcatacgagaaactccattttcccttttctttgtcagcttga

SEQ ID NO: 10 - DNA, 1935 bp, Arabidopsis thaliana
atggtaaatccgggtcacggaagaggacccgattcgggtactgctgctggtgggtcaaactccgacccgtttcctgcgaatcttcgagttcttgtcgttgatgatgatccaacttgtctcatgatcttagagaggatgcttatgacttgtctctacagagagcagagagcgcattgtctctgcttcggaagaacaaagaatggttttgatattgtcattagtaatgttcatatgcctgacatggatggtttcaagctccttgaacacgttggtttagagatggatttacctgttatcaatctgaatgttttgaaacctttggttatagtgatgtctgcggatgattcgaagagcgttgtgttgaaaggagtgactcacggtgcagttgattacctcatcaaaccggtacgtattgaggctttgaagaatatatggcaacatgtggtgcggaagaagcgtaacgagtggaatgtttctgaacattctggaggaagtattgaagatactggcggtgacagggacaggcagcagcagcatagggaggatgctgataacaactcgtcttcagttaatgaagggaacgggaggagctcgaggaagcggaaggaagaggaagtagatgatcaaggggatgataaggaagactcatcgagtttaaagaaaccacgcgtggtttggtctgttgaattgcatcagcagtttgttgctgctgtgaatcagctaggcgttgacagtgagttaaaaacttgcttgcttatgcatttgtgtgtgtcgattggtaacattgtggaattccagaagtatcggatatatctgagacggcttggaggagtatcgcaacaccaaggaaatatgaaccattcgtttatgactggtcaagatcagagttttggacctctttcttcgttgaatggatttgatcttcaatctttagctgttactggtcagctccctcctcagagccttgcacagcttcaagcagctggtcttggccggcctacactcgctaaaccagggatgtcggtttctccccttgtagatcagagaagcatcttcaactttgaaaacccaaaaataagatttggagacggacatggtcagacgatgaacaatggaaatttgcttcatggtgtcccaacgggtagtcacatgcgtctgcgtcctggacagaatgttcagagcagcggaatgatgttgccagtagcagaccagctacctcgaggaggaccatcgatgctaccatccctcgggcaacagccgatattgtcaagcagcgtttcaagaagaagcgatctcactggtgcgctggcggttagaaacagtatccccgagaccaacagcagagtgttaccaactactcactcggtcttcaataacttccccgcggatctacctcgcagcagcttcccgttggcaagtgccccagggatttcagttccagtatcagtttcttaccaagaagaggtcaacagctcggatgcaaaaggaggttcatcagctgctactgctggatttggtaacccaagctacgacatatttaacgattttccgcagcaccaacagcacaacaagaacatcagcaataaactaaacgattgggatctgcggaatatgggattggtcttcagttccaatcaggacgcagcaactgcaaccgcaaccgcagcattttccacttcggaagcatactcttcgtcttctacgcagagaaaaagacgggaaacggacgcaacagttgtgggtgagcatgggcagaacctgcagtcaccgagccggaatctgtatcatctgaaccacgtttttatggacggtggttcagtcagagtgaagtcagaaagagtggcggagacagtgacttgtcctccagcaaatacattgtttcacgagcagtataatcaagaagatctgatgagcgcatttctcaaacaggtttga

SEQ ID NO: 11 - DNA, 780 bp, Artificial Sequence
  derived from Arabidopsis thaliana
atggccagagacggtggtgtttcttgtttacgaaggtcggagatgatgagcgtcggtggtatcggaggaattgaatctgcgccgttggatttagatgaagttcatgtcttagccgttgatgacagtctcgttgatcgtattgtcatcgagagattgcttcgtattacttcctgcaaagttacggcggtagatagtggatggcgtgctctggaatttctagggttagataatgagaaagcttctgctgaattcgatagattgaaagttgatttgatcatcactaattactgtatgcctggaatgactggttatgagcttctcaagaagattaaggaatcgtccaatttcagagaagttccggttgtaatcatgtcgtcggagaatgtattgaccagaatcgacagatgccttgaggaaggtgctcaagatttcttattgaaaccggtgaaactcgccgacgtgaaacgtctgagaagtcatttaactaaagacgttaaactttccaacggaaacaaacggaagcttccggaagattctagttccgttaactcttcgcttcctccaccgtcacctccgttgactatctcgcctgaatcgtcgccgccgttgactgtttcgactgaatcgtcggattcgtctccgccgttatctccggtggagatcttttccacctcgccactttcatctccgatagacgatgaagatgatgacgtgttgacgtcgtcgtcggaggaatcgccgattcgacggcagaagatgaggagtcccggattagattag

SEQ ID NO: 12 - DNA, 942 bp, Arabidopsis thaliana
atcctccaacattcgttgatcaatggctgaagttatgctaccgaggaagatggagattctcaaccattcttcaaagtttggatcaccggatcctcttcatgttcttgccgtcgacgacagtcacgttgatcgtaaattcatcgagcgtttgctcagagtatcttcttgcaaagttactgttgttgatagtgcaacaagagctctccaataccttggtttggatgttgaggaaaaatcagtcggttttgaggatttgaaggttaatttaattatgactaattactcaatgcccggtatgactggatatgaactcttgaagaagatcaaagaatcctcagcttttagagaagtaccggtggtaattatgtcctccgagaacattttgcctcgtattgatagatgtcttgaagaaggggctgaagatttcttactgaagccggtaaaactctcggatgtaaaaagattaagagattctctaatgaaggttgaagatttgtctttcacaaagagtattcagaagagagagctagaaacagagaatgtctaccctgttcactcgcagctcaaacgcgcaaagatctgagctctccgatgcaaattccgtgactggatcttagaaaaagccttttcatatacaggaagcttcaagactcaatgcacttgagctacactgatgattctcgccagcttaatgacctatacaattttccaacccctatggcctttttcgctgattgaattcagtgggtttattcatatagtagtaatgtaaatggtatagagatgagcaaaagctatattatatattatattcattagaggggtcatagcttttttccttacaagagatttgtaagctggcttaatagaagttgttaagaagcctcactttatgtgaataaatgattttgttagctaatcataaacaatcagaagtttcct

SEQ ID NO: 13 - DNA, 3677 bp, Arabidopsis thaliana
aattattcactcaaattcacaaagggtttcgactttgctccgaggaagaagataatatgaaaagagctttttagggtttatcattctccttgactttgcaaaacgtgaaataagcttcttccttggaggactaatcaagacagaaatctgttcctctaaaaacgatcgccgttctagttcactcaaatgatggtgaaagttacaaagcttgtggcttcacgtccaattgtggtcttttgcgtcctggcattcctggtggttgttttcgagtgcatttggatctcaaattggcgaacaacaacggagaacctagtcaaagaggtcgcttcatttaccgaagatctccggacaagtctagtttcggagattgaaaacatcggaaaatttacatatgctaagacaaacttatctacgatcggtttagcgagagttatagattcttatatcaccaacaacgacactggttttacagagattcaaacacagatcgcaccattgttgtttgtagcttattcaacgatccttcaagtctcacaagtttcgtacatcagtagggacggtctcatgttttcttacattgcagaatcaaacacaagtgtcgctgtttttgccaattcctcgtcgaattcaagtcgtggagactacacttggtacactcaaaccgtggatcagttaactggtcgtcttaacgggaactcaacgaaatctcagtcgttagatgtaacccatacagattggttccaagcagcacagagtaataactacactacagcctttgtaggaacgagcttgggaggagaagataacgagactctaatacagagcgtggttagcttgtacagcaagaaaggtcttgtttctttagggtttccggttaagactttaaccgaagttttgaacagtttgaatctacacggcgaagagctttacatgtggacaaaggacgggacggtgcttgttcgtgaaggttcactgaatgattctttcttcatctccaatggctcgatttgcttcggtagagaatcgaactccctctggtctcaatgcatccctgaaaattgcagttccagtggctacgaggtggagatcaaaagattaagataccaagctttttgctctgttattgaagtttcgggcgttcctctgagatacacactcatgtttcccaacaaaggaggagcaacacgcatcaagcaccaagcggaaaaggcaaaatatcaacttattgtggttatgatatttcttggcttcggttggcctgtatggtttgtgtggtttatgatgcaagcaacaaggagagagatgcatatgcgtgcaacgctgataaaccaaatggaagcgacacaacaagctgagagaaagagcatgaacaagagtcaagcatttgcaaatgctagccacgatattagaggtgcccttgcagggatgaaaggtctgattgatatatgtcgtgatggagttaaacctggctccgacgtagacaccactctcaaccaagtgaatgtttgcgccaaggatttggttgctctgctcaactctgttttggacatgagcaaaatcgaaagcgggaagatgcagttagtggaagaagatttcaacttgtcgaaacttcttgaagacgtcatcgatttttaccatcccgttgcgatgaagaaaggggttgatgtagttttggatccgcacgatggctcggttttcaaattctcgaatgtacgaggggatagtggcagactgaagcagattctgaacaatcttgttagcaatgctgtcaagttcaccgtcgacgggcacattgcggtaagagcttgggctcagaggccaggttccaatagctctgtggtccttgcatcatatcctaaaggtgtgtccaagtttgtaaagagtatgttctgcaagaataaagaagagtcatcaacctacgagacagaaatatcgaattccataagaaacaatgcaaacacgatggagtttgtgtttgaagtggatgatactggtaaagggatacctatggagatgcgtaagtcggtatttgaaaactatgttcaagtaagagaaacagctcaaggacaccaaggaactggtttagggctcgggattgtgcagtctttggtaagattaatgggaggggagataagaatcaccgacaaggccatgggagagaaaggaacatgtttccaattcaatgttttattgacaacattagagtctcctccagtgagtgacatgaaagtgagacaggagatcgaagcaggaggcgattatgtatccacgccaaacctcgggctgactataaacacttcacttggaggtagcatgaatatacgtaacctgagtcctagattcaacaactgtctcagctcaagtccaaagcaagaaggttctagagtggttcttctattgaaaaatgaagaacgcagaagagttacagagaaatacataaagaatcttgggattaaagttacagtggtggagaaatgggagcatttgagttatgctttggagagactttttggattttcacctcagagttccatgggaagagcagagtgtagtttgtcatgtccgagctcaagggagttacctttcattggcatggacggtattgattcaagaagccaacttcctaaaaggagaagcatcagtttctctgcagttgtccttttggtgattgatgcaaaaactggaccattttttgagctgtgcgatattgtcaaacagtttcgtagaggcttgccccatggaatatcctgtaaagttgtttggcttaacgaatcgagcactcgtgtaagtgagagaggggacattagttgttcgagacctttgcacggatcgcgtcttatggaagtcttgaagatgttgcctgaatttggaggaactgtgctaaaagaaccacctactgagctgcaaagggaatcattgctgagacattcttttgttgcagagagatcaccaaaacataaagtccaagaagaggggccaagctcaatgtttaacaaaaaattaggtaagaggataatggcatcaacagattcagagagtgagactagggtcaagtcagtgcgtaccggtcgaaagcctattgggaacccagaggacgagcaagagacttccaagccgagtgacgatgaattcttaagaggaaagagagttcttgtggtcgatgataactttatatcacgtaaagttgcaacaggaaagctgaagaagatgggagtctcagaggtcgaacaatgcgacagtgggaaagaagctttgagattagtcactgaagggcttacacaaagagaagaacaaggttcagtagataaacttccgtttgactacatattcatggactgccaaatgccagaaatggatggctatgaagcaactagagagattaggaaagtggagaaaagttatggggtgcgtacaccaattatagctgtatctggtcatgatcctggttcagaggaagcaagagaaaccattcaagctggaatggacgccttcttagataaaagcttgaatcaacttgcaaacgtcattagagaaatcgaaagcaaacgtcactagtattgtataagtagacagcacgcagctttgtgctactaccattagtgttttagacaatacttttttagtatgacgatgtcatagcagttttcttctaagtttatttaaagtccatttcagat

SEQ ID NO: 14 - DNA, 3677 bp, Artificial Sequence
  derived from Arabidopsis thaliana
aattattcactcaaattcacaaagggtttcgactttgctccgaggaagaagataatatgaaaagagctttttagggtttatcattctccttgactttgcaaaacgtgaaataagcttcttccttggaggactaatcaagacagaaatctgttcctctaaaaacgatcgccgttctagttcactcaaatgatggtgaaagttacaaagcttgtggcttcacgtccaattgtggtcttttgcgtcctggcattcctggtggttgttttcgagtgcatttggatctcaaattggcgaacaacaacggagaacctagtcaaagaggtcgcttcatttaccgaagatctccggacaagtctagtttcggagattgaaaacatcggaaaatttacatatgctaagacaaacttatctacgatcggtttagcgagagttatagattcttatatcaccaacaacgacactggttttacagagattcaaacacagatcgcaccattgttgtttgtagcttattcaacgatccttcaagtctcacaagtttcgtacatcagtagggacggtctcatgttttcttacattgcagaatcaaacacaagtgtcgctgtttttgccaattcctcgtcgaattcaagtcgtggagactacacttggtacactcaaaccgtggatcagttaactggtcgtcttaacgggaactcaacgaaatctcagtcgttagatgtaacccatacagattggttccaagcagcacagagtaataactacactacagcctttgtaggaacgagcttgggaggagaagataacgagactctaatacagagcgtggttagcttgtacagcaagaaaggtcttgtttctttagggtttccggttaagactttaaccgaagttttgaacagtttgaatctacacggcgaagagctttacatgtggacaaaggacgggacggtgcttgttcgtgaaggttcactgaatgattctttcttcatctccaatggctcgatttgcttcggtagagaatcgaactccctctggtctcaatgcatccctgaaaattgcagttccagtggctacgaggtggagatcaaaagattaagataccaagctttttgctctgttattgaagtttcgggcgttcctctgagatacacactcatgtttcccaacaaaggaggagcaacacgcatcaagcaccaagcggaaaaggcaaaatatcaacttattgtggttatgatatttcttggcttcggttggcctgtatggtttgtgtggtttatgatgcaagcaacaaggagagagatgcatatgcgtgcaacgctgataaaccaaatggaagcgacacaacaagctgagagaaagagcatgaacaagagtcaagcatttgcaaatgctagccaagatattagaggtgcccttgcagggatgaaaggtctgattgatatatgtcgtgatggagttaaacctggctccgacgtagacaccactctcaaccaagtgaatgtttgcgccaaggatttggttgctctgctcaactctgttttggacatgagcaaaatcgaaagcgggaagatgcagttagtggaagaagatttcaacttgtcgaaacttcttgaagacgtcatcgatttttaccatcccgttgcgatgaagaaaggggttgatgtagttttggatccgcacgatggctcggttttcaaattctcgaatgtacgaggggatagtggcagactgaagcagattctgaacaatcttgttagcaatgctgtcaagttcaccgtcgacgggcacattgcggtaagagcttgggctcagaggccaggttccaatagctctgtggtccttgcatcatatcctaaaggtgtgtccaagtttgtaaagagtatgttctgcaagaataaagaagagtcatcaacctacgagacagaaatatcgaattccataagaaacaatgcaaacacgatggagtttgtgtttgaagtggatgatactggtaaagggatacctatggagatgcgtaagtcggtatttgaaaactatgttcaagtaagagaaacagctcaaggacaccaaggaactggtttagggctcgggattgtgcagtctttggtaagattaatgggaggggagataagaatcaccgacaaggccatgggagagaaaggaacatgtttccaattcaatgttttattgacaacattagagtctcctccagtgagtgacatgaaagtgagacaggagatcgaagcaggaggcgattatgtatccacgccaaacctcgggctgactataaacacttcacttggaggtagcatgaatatacgtaacctgagtcctagattcaacaactgtctcagctcaagtccaaagcaagaaggttctagagtggttcttctattgaaaaatgaagaacgcagaagagttacagagaaatacataaagaatcttgggattaaagttacagtggtggagaaatgggagcatttgagttatgctttggagagactttttggattttcacctcagagttccatgggaagagcagagtgtagtttgtcatgtccgagctcaagggagttacctttcattggcatggacggtattgattcaagaagccaacttcctaaaaggagaagcatcagtttctctgcagttgtccttttggtgattgatgcaaaaactggaccattttttgagctgtgcgatattgtcaaacagtttcgtagaggcttgccccatggaatatcctgtaaagttgtttggcttaacgaatcgagcactcgtgtaagtgagagaggggacattagttgttcgagacctttgcacggatcgcgtcttatggaagtcttgaagatgttgcctgaatttggaggaactgtgctaaaagaaccacctactgagctgcaaagggaatcattgctgagacattcttttgttgcagagagatcaccaaaacataaagtccaagaagaggggccaagctcaatgtttaacaaaaaattaggtaagaggataatggcatcaacagattcagagagtgagactagggtcaagtcagtgcgtaccggtcgaaagcctattgggaacccagaggacgagcaagagacttccaagccgagtgacgatgaattcttaagaggaaagagagttcttgtggtcgatgataactttatatcacgtaaagttgcaacaggaaagctgaagaagatgggagtctcagaggtcgaacaatgcgacagtgggaaagaagctttgagattagtcactgaagggcttacacaaagagaagaacaaggttcagtagataaacttccgtttgactacatattcatggactgccaaatgccagaaatggatggctatgaagcaactagagagattaggaaagtggagaaaagttatggggtgcgtacaccaattatagctgtatctggtcatgatcctggttcagaggaagcaagagaaaccattcaagctggaatggacgccttcttagataaaagcttgaatcaacttgcaaacgtcattagagaaatcgaaagcaaacgtcactagtattgtataagtagacagcacgcagctttgtgctactaccattagtgttttagacaatacttttttagtatgacgatgtcatagcagttttcttctaagtttatttaaagtccatttcagat

SEQ ID NO: 15 - DNA, 3677 bp, Artificial Sequence
  derived from Arabidopsis thaliana
aattattcactcaaattcacaaagggtttcgactttgctccgaggaagaagataatatgaaaagagctttttagggtttatcattctccttgactttgcaaaacgtgaaataagcttcttccttggaggactaatcaagacagaaatctgttcctctaaaaacgatcgccgttctagttcactcaaatgatggtgaaagttacaaagcttgtggcttcacgtccaattgtggtcttttgcgtcctggcattcctggtggttgttttcgagtgcatttggatctcaaattggcgaacaacaacggagaacctagtcaaagaggtcgcttcatttaccgaagatctccggacaagtctagtttcggagattgaaaacatcggaaaatttacatatgctaagacaaacttatctacgatcggtttagcgagagttatagattcttatatcaccaacaacgacactggttttacagagattcaaacacagatcgcaccattgttgtttgtagcttattcaacgatccttcaagtctcacaagtttcgtacatcagtagggacggtctcatgttttcttacattgcagaatcaaacacaagtgtcgctgtttttgccaattcctcgtcgaattcaagtcgtggagactacacttggtacactcaaaccgtggatcagttaactggtcgtcttaacgggaactcaacgaaatctcagtcgttagatgtaacccatacagattggttccaagcagcacagagtaataactacactacagcctttgtaggaacgagcttgggaggagaagataacgagactctaatacagagcgtggttagcttgtacagcaagaaaggtcttgtttctttagggtttccggttaagactttaaccgaagttttgaacagtttgaatctacacggcgaagagctttacatgtggacaaaggacgggacggtgcttgttcgtgaaggttcactgaatgattctttcttcatctccaatggctcgatttgcttcggtagagaatcgaactccctctggtctcaatgcatccctgaaaattgcagttccagtggctacgaggtggagatcaaaagattaagataccaagctttttgctctgttattgaagtttcgggcgttcctctgagatacacactcatgtttcccaacaaaggaggagcaacacgcatcaagcaccaagcggaaaaggcaaaatatcaacttattgtggttatgatatttcttggcttcggttggcctgtatggtttgtgtggtttatgatgcaagcaacaaggagagagatgcatatgcgtgcaacgctgataaaccaaatggaagcgacacaacaagctgagagaaagagcatgaacaagagtcaagcatttgcaaatgctagccacgatattagaggtgcccttgcagggatgaaaggtctgattgatatatgtcgtgatggagttaaacctggctccgacgtagacaccactctcaaccaagtgaatgtttgcgccaaggatttggttgctctgctcaactctgttttggacatgagcaaaatcgaaagcgggaagatgcagttagtggaagaagatttcaacttgtcgaaacttcttgaagacgtcatcgatttttaccatcccgttgcgatgaagaaaggggttgatgtagttttggatccgcacgatggctcggttttcaaattctcgaatgtacgaggggatagtggcagactgaagcagattctgaacaatcttgttagcaatgctgtcaagttcaccgtcgacgggcacattgcggtaagagcttgggctcagaggccaggttccaatagctctgtggtccttgcatcatatcctaaaggtgtgtccaagtttgtaaagagtatgttctgcaagaataaagaagagtcatcaacctacgagacagaaatatcgaattccataagaaacaatgcaaacacgatggagtttgtgtttgaagtggatgatactggtaaagggatacctatggagatgcgtaagtcggtatttgaaaactatgttcaagtaagagaaacagctcaaggacaccaaggaactggtttagggctcgggattgtgcagtctttggtaagattaatgggaggggagataagaatcaccgacaaggccatgggagagaaaggaacatgtttccaattcaatgttttattgacaacattagagtctcctccagtgagtgacatgaaagtgagacaggagatcgaagcaggaggcgattatgtatccacgccaaacctcgggctgactataaacacttcacttggaggtagcatgaatatacgtaacctgagtcctagattcaacaactgtctcagctcaagtccaaagcaagaaggttctagagtggttcttctattgaaaaatgaagaacgcagaagagttacagagaaatacataaagaatcttgggattaaagttacagtggtggagaaatgggagcatttgagttatgctttggagagactttttggattttcacctcagagttccatgggaagagcagagtgtagtttgtcatgtccgagctcaagggagttacctttcattggcatggacggtattgattcaagaagccaacttcctaaaaggagaagcatcagtttctctgcagttgtccttttggtgattgatgcaaaaactggaccattttttgagctgtgcgatattgtcaaacagtttcgtagaggcttgccccatggaatatcctgtaaagttgtttggcttaacgaatcgagcactcgtgtaagtgagagaggggacattagttgttcgagacctttgcacggatcgcgtcttatggaagtcttgaagatgttgcctgaatttggaggaactgtgctaaaagaaccacctactgagctgcaaagggaatcattgctgagacattcttttgttgcagagagatcaccaaaacataaagtccaagaagaggggccaagctcaatgtttaacaaaaaattaggtaagaggataatggcatcaacagattcagagagtgagactagggtcaagtcagtgcgtaccggtcgaaagcctattgggaacccagaggacgagcaagagacttccaagccgagtgacgatgaattcttaagaggaaagagagttcttgtggtcgatgataactttatatcacgtaaagttgcaacaggaaagctgaagaagatgggagtctcagaggtcgaacaatgcgacagtgggaaagaagctttgagattagtcactgaagggcttacacaaagagaagaacaaggttcagtagataaacttccgtttgactacatattcatgaactgccaaatgccagaaatggatggctatgaagcaactagagagattaggaaagtggagaaaagttatggggtgcgtacaccaattatagctgtatctggtcatgatcctggttcagaggaagcaagagaaaccattcaagctggaatggacgccttcttagataaaagcttgaatcaacttgcaaacgtcattagagaaatcgaaagcaaacgtcactagtattgtataagtagacagcacgcagctttgtgctactaccattagtgttttagacaatacttttttagtatgacgatgtcatagcagttttcttctaagtttatttaaagtccatttcagat

SEQ ID NO: 16 - DNA, 3531 bp, Arabidopsis thaliana
atgtctataacttgtgagctcttgaatcttacttcaaagaaagctaagaagtcgtcgagcagtgacaagaaatggctaaagaagcctctcttcttcctgattttgtgtggctctttggtaattgttttggttatgttcttacggttaggtagaagtcagaaggaggagacagattcttgtaatggagaagagaaagtgttgtatagacatcaaaatgtcacaagaagtgagattcatgatttggtctctttgttctctgattcagatcaggtaacatcctttgaatgtcataaggaatcaagccctggaatgtggacaaactatggtattacatgttccctgagtgtgcgttctgataaacaagagactagagggcttccctggaatcttggcttaggacattctatctcatcaacatcttgtatgtgtggtaatcttgaaccgattttacagcaacctgaaaaccttgaggaagaaaaccatgaagaagggctggagcagggtttgtcatcgtatttaagaaatgcatggtggtgtctaatccttggtgtgttagtgtgccataagatttatgtatctcattctaaagcacgaggtgagaggaaagagaaagtacatctgcaagaggctttagctccaaagaagcagcaacaacgtgctcagacttcttctagaggggctggaagatggaggaagaatatccttctccttggtattttaggaggagtttccttctctgtttggtggttttgggacactaatgaggagatcataatgaaaaggagggagactttggcaaacatgtgtgacgaacgagcacgtgttttacaagatcagttcaatgttagcttgaaccatgttcatgccttgtctattcttgtatctacatttcatcatggtaaaatcccatctgccattgatcagagaacatttgaagaatatactgagagaacaaactttgagaggccacttactagtggtgtagcgtatgctttgaaagtcccacactcagaaagagagaaatttgaaaaggagcatggatgggcaataaagaaaatggaaactgaggaccagacagttgtacaagattgtgttcctgaaaactttgatcccgcaccgattcaagacgaatacgcgccagttatatttgctcaagaaactgtttcccatattgtatcggtcgacatgatgtctggagaagaagaccgtgaaaacatcttacgggcaagggcatcaggaaaaggagtgttaacatctccatttaagcttcttaagtcaaatcatcttggtgttgtgttgacctttgctgtctatgacacgagcctaccgcctgatgctacagaagaacagcgtgttgaagcaactattgggtaccttggtgcatcatatgatatgccatcgctggtggagaaacttcttcaccaacttgccagcaaacagacaattgctgtggatgtttacgacacaactaacacttcaggtctaataaaaatgtatggctcagaaattggggatataagtgagcagcatataagtagccttgattttggtgatccatcaaggaaccatgagatgcattgcaggtttaagcataaacttcccattccctggacagcgataacaccgtcgatcttagttctggttattacttttcttgttggttatattttatatgaagccatcaaccgaattgcgacagttgaagaggattgtcagaagatgagggaactcaaagctcgtgctgaggccgctgacattgcaaagtcacagttcctagcaactgtttctcatgagatacggactccgatgaatggagttttaggaatgctgaaaatgctgatggacaccgatcttgatgcgaagcagatggactatgcgcaaactgctcatggcagtgggaaggatcttacatcactaataaatgaggttcttgatcaggcaaagattgaatccggaaggctcgagcttgaaaatgtgccttttgatatgcgttttattcttgataatgtttcatctctcctctctggcaaggcaaatgaaaaaggaattgagttggccgtttatgtttctagtcaagttcctgatgttgtagtcggtgatccgagtcggttccggcagatcattacaaacctggttggaaactcaatcaaattcacacaggaaaggggacacatatttatctcagtgcaccttgcagatgaggtaaaggagcctcttactattgaagacgcagtgctaaaacagcgactagctttaggatgcagcgagtccggtgagacagttagcgggtttcctgcggtaaatgcatggggaagctggaagaatttcaagacatgttacagtactgagagtcagaattctgatcaaatcaaattgctagttacagtggaggacactggagttggcatacctgtggatgcacaaggccgaatcttcacaccttttatgcaagccgacagttccacatcgcggacttatggtggaactggcataggtttgagtataagcaaacgtttggttgaactcatgcaaggagagatggggtttgtgagtgagcccgggataggcagtactttttcatttactggagttttcgggaaagcagaaacaaatacgtcgattactaagctggaacgattcgatctagctattcaggagtttacaggattgagagcattagttattgataacagaaacattagagcagaggtcaccaggtacgaacttcggagactgggaatatctgcagacattgtttcaagtctgagaatggcatgcacttgttgtatcagcaaattagaaaatttggctatgattctaatagacaaagacgcctggaacaaggaagaattttcagtacttgacgagttgtttacccgaagcaaagtaacctttacaagagtcccaaagatttttcttttggcaacttctgcaactcttactgagcgcagtgagatgaagtctactggtctcatcgatgaggtggtgataaagcctcttcggatgagtgtcttaatatgttgcttgcaagaaacccttgtcaatggcaagaagaggcaaccgaacagacagcgaagaaatcttggacacttgctaagagaaaaacagattctggttgtggatgataatcttgtgaacagacgagttgcagaaggtgcacttaagaaatatggagctattgttacatgcgttgagagtggcaaagctgcattggcaatgcttaagccgcctcataacttcgatgcttgcttcatggatctccagatgcctgaaatggatggatttgaagcgacaaggagagtccgtgagctggagagggaaatcaataagaaaatagcttctggagaagtttcagctgaaatgttctgtaaatttagtagttggcacgtcccgatattagcaatgacagcagatgttattcaggctactcatgaagaatgcatgaaatgtggaatggatggttatgtatcaaaaccgtttgaagaggaagtgctctacacagcggtagcaagattctttgaaccttgttaa

SEQ ID NO: 17 - DNA, 1503 bp, Arabidopsis thaliana
atggctccagtaataaagctggtcttaggttcagttgcctttgccattttctggatattagcagtgtttccctctgtaccattcctaccaattggtcgaaccgccggttctctgttcggtgcaatgctaatggttatcttccaagtgataaccccggaacaagcttatgcagccattgatctcccgatactcggtcttctctttggaacgatggttgttagtatataccttgagagagctgatatgttcaagtacttaggcacattgctttcgtggaaaagcagaggtccgaaagacttgctatgtagagtctgtcttgtttccgctgtttcaagcgctctgtttactaacgatacatcttgtgtggttttaaccgagtttgtgttgaagatcgctaggcaaaagaatctcccgcctcacccgtttttgctcgctttagccacgagtgcgaatattggttcttctgctactcccattgggaatccgcagaatcttgttattgcggttcaaagcaagatcccgttttgggagtttcttcttggtgtttttcctgcgatgattgttggtattaccgttaacgctatgcttcttctcggtatgtattggcggttattgtcagatcataaagaggatgaagaagaagtacaaaatgctgattctgaagttgttgcccaagaagatgtccaatctcataggttctcgccagctacattttcgccagtctcatccgaagattcgaacttgagaatggatgccgccgagactctcagaaacagagcgggttcagcgggtgagagcgagttaataagctgcaattcaaatgcgtcgagagaacaacacaacgacgcagagtctcaaggagagagcaataataccaacaatatgttccagaccaagagatggagaagagttttatggaaatcaagtgtttatttcatcacattaggaatgctaatatctctgcttatgggtttaaacatgtcgtggaccgcaattaccgcggctctagcactcgtggttcttgatttcaaagacgcaaggccgtctctcgagaaggtatcgtattcgcttttgatctttttctgcgggatgttcataaccgttgatggattcaacaaaactggtatccctaccgctctatgggacctaatggagccatatgccaaaattgatcaagccaaaggaatcgcggttctagcagttgtgattcttgtcctctccaatgtagcctctaatgtaccaaccgtgcttttgttgggagcaagagtggcggcatcagcgatggggagggaggaggagaagaaggcgtggttgttgctggcgtgggtgagcacagtggctggaaacttgacgttgcttggttcagcagcaaacctgatagtgtgtgagcaagctcgtagagcagtgagccatggatacactctcactttcactaaacatttcaaattcggtttgccttcaacactcatcgtcactgcgattggtctcttccttatcaagtaa

SEQ ID NO: 18 - DNA, 1803 bp, Artificial Sequence
  derived from Arabidopsis thaliana
atgcacatagtaaaagtcgaagatgatttccatgaaatgcaagagcttaaagttcgagcagaagctgctgatgtcgctaaatcgcagtttcttgctaccgtgtctcacgagatcaggacaccaatgaatggcattctcggaatgcttgctatgctcctagatacagaactaagctcgacacagagagattacgctcaaaccgctcaagtatgtggtaaagctttgattgcattgataaatgaggttcttgatcgcgccaagattgaagctggaaagctggagttggaatcagtaccatttgatatccgttcaatattggatgatgtcctttctctattctctgaggagtcaaggaacaaaagcattgagctcgcggttttcgtttcagacaaagtaccagagatagtcaaaggagattcagggagatttagacagataatcataaaccttgttggaaattcggttaaattcacagagaaaggacatatctttgttaaagtccatcttgcggaacaatcaaaagatgaatctgaaccgaaaaatgcattgaatggtggagtcaatgttaggcttatggtttcaatcgaagacacgggtattggaatccctttagttgcgcaaggccgtgtgtttatgccgtttatgcaagcagatagctcgacttcaagaaactatggaggtactggtattggtttgagtataagcaagtgtcttgttgaacttatgcgtggtcagataaatttcataagccggcctcatattggaagcacgttctggttcacggctgttttagagaaatgcgataaatgcagtgcgattaaccatatgaagaaacctaatgtggaacacttgccttctacttttaaaggaatgaaagctatagttgttgatgctaagcctgttagagctgctgtgactagataccatatgaaaagactcggaatcaatgttgatgtcgtgacaagtctcaaaaccgctgttgttgcagctgctgcgtttgaaagaaacggttctcctctcccaacaaaaccgcaacttgatatgatcttagtagagaaagattcatggatttcaactgaagataatgactcagagattcgtttattgaattcaagaaccaacggaaacgttcatcacaagtctccgaaactagctctattcgcaacaaacatcacaaattcggagttcgacagagctaaatccgcaggatttgcagatacggtaataatgaaaccgttaagagcaagcatgattggggcgtgtctgcaacaagttctcgagctgagaaaaacaagacaacaacatccagaaggatcatcacccgcaactctcaagagcttgcttacagggaagaagattcttgtggttgatgataatatagttaacaggagagtagctgcaggagctctcaagaaatttggagcagaagtggtttgtgcagagagtggtcaagttgctttgggtttgcttcagattccacacactttcgatgcttgcttcatggatattcaaatgccacagatggacggaatgatggagaaggaaactaaagagaagacaaatctcgaatggcatttaccgattctagcgatgactgcggatgtgatacacgcgacctacgaggaatgtctgaaaagtgggatggatggttacgtctccaaaccttttgaagaagagaatctctataaatccgttgccaaatcattcaaacctaatcctatctcaccttcccggaagagtggaccgattctgctgattgatatgcattttggtttctgtacatacagtaggttcacaatctag

SEQ ID NO: 19 - DNA, 1803 bp, Artificial Sequence
  derived from Arabidopsis thaliana
atgcacatagtaaaagtcgaagatgatttccatgaaatgcaagagcttaaagttcgagcagaagctgctgatgtcgctaaatcgcagtttcttgctaccgtgtctcaagagatcaggacaccaatgaatggcattctcggaatgcttgctatgctcctagatacagaactaagctcgacacagagagattacgctcaaaccgctcaagtatgtggtaaagctttgattgcattgataaatgaggttcttgatcgcgccaagattgaagctggaaagctggagttggaatcagtaccatttgatatccgttcaatattggatgatgtcctttctctattctctgaggagtcaaggaacaaaagcattgagctcgcggttttcgtttcagacaaagtaccagagatagtcaaaggagattcagggagatttagacagataatcataaaccttgttggaaattcggttaaattcacagagaaaggacatatctttgttaaagtccatcttgcggaacaatcaaaagatgaatctgaaccgaaaaatgcattgaatggtggagtcaatgttaggcttatggtttcaatcgaagacacgggtattggaatccctttagttgcgcaaggccgtgtgtttatgccgtttatgcaagcagatagctcgacttcaagaaactatggaggtactggtattggtttgagtataagcaagtgtcttgttgaacttatgcgtggtcagataaatttcataagccggcctcatattggaagcacgttctggttcacggctgttttagagaaatgcgataaatgcagtgcgattaaccatatgaagaaacctaatgtggaacacttgccttctacttttaaaggaatgaaagctatagttgttgatgctaagcctgttagagctgctgtgactagataccatatgaaaagactcggaatcaatgttgatgtcgtgacaagtctcaaaaccgctgttgttgcagctgctgcgtttgaaagaaacggttctcctctcccaacaaaaccgcaacttgatatgatcttagtagagaaagattcatggatttcaactgaagataatgactcagagattcgtttattgaattcaagaaccaacggaaacgttcatcacaagtctccgaaactagctctattcgcaacaaacatcacaaattcggagttcgacagagctaaatccgcaggatttgcagatacggtaataatgaaaccgttaagagcaagcatgattggggcgtgtctgcaacaagttctcgagctgagaaaaacaagacaacaacatccagaaggatcatcacccgcaactctcaagagcttgcttacagggaagaagattcttgtggttgatgataatatagttaacaggagagtagctgcaggagctctcaagaaatttggagcagaagtggtttgtgcagagagtggtcaagttgctttgggtttgcttcagattccacacactttcgatgcttgcttcatggatattcaaatgccacagatggacggaatgatggagaaggaaactaaagagaagacaaatctcgaatggcatttaccgattctagcgatgactgcggatgtgatacacgcgacctacgaggaatgtctgaaaagtgggatggatggttacgtctccaaaccttttgaagaagagaatctctataaatccgttgccaaatcattcaaacctaatcctatctcaccttcccggaagagtggaccgattctgctgattgatatgcattttggtttctgtacatacagtaggttcacaatctag

SEQ ID NO: 20 - DNA, 1803 bp, Artificial Sequence
  derived from Arabidopsis thaliana
atgcacatagtaaaagtcgaagatgatttccatgaaatgcaagagcttaaagttcgagcagaagctgctgatgtcgctaaatcgcagtttcttgctaccgtgtctcacgagatcaggacaccaatgaatggcattctcggaatgcttgctatgctcctagatacagaactaagctcgacacagagagattacgctcaaaccgctcaagtatgtggtaaagctttgattgcattgataaatgaggttcttgatcgcgccaagattgaagctggaaagctggagttggaatcagtaccatttgatatccgttcaatattggatgatgtcctttctctattctctgaggagtcaaggaacaaaagcattgagctcgcggttttcgtttcagacaaagtaccagagatagtcaaaggagattcagggagatttagacagataatcataaaccttgttggaaattcggttaaattcacagagaaaggacatatctttgttaaagtccatcttgcggaacaatcaaaagatgaatctgaaccgaaaaatgcattgaatggtggagtcaatgttaggcttatggtttcaatcgaagacacgggtattggaatccctttagttgcgcaaggccgtgtgtttatgccgtttatgcaagcagatagctcgacttcaagaaactatggaggtactggtattggtttgagtataagcaagtgtcttgttgaacttatgcgtggtcagataaatttcataagccggcctcatattggaagcacgttctggttcacggctgttttagagaaatgcgataaatgcagtgcgattaaccatatgaagaaacctaatgtggaacacttgccttctacttttaaaggaatgaaagctatagttgttgatgctaagcctgttagagctgctgtgactagataccatatgaaaagactcggaatcaatgttgatgtcgtgacaagtctcaaaaccgctgttgttgcagctgctgcgtttgaaagaaacggttctcctctcccaacaaaaccgcaacttgatatgatcttagtagagaaagattcatggatttcaactgaagataatgactcagagattcgtttattgaattcaagaaccaacggaaacgttcatcacaagtctccgaaactagctctattcgcaacaaacatcacaaattcggagttcgacagagctaaatccgcaggatttgcagatacggtaataatgaaaccgttaagagcaagcatgattggggcgtgtctgcaacaagttctcgagctgagaaaaacaagacaacaacatccagaaggatcatcacccgcaactctcaagagcttgcttacagggaagaagattcttgtggttgatgataatatagttaacaggagagtagctgcaggagctctcaagaaatttggagcagaagtggtttgtgcagagagtggtcaagttgctttgggtttgcttcagattccacacactttcgatgcttgcttcatgaatattcaaatgccacagatggacggaatgatggagaaggaaactaaagagaagacaaatctcgaatggcatttaccgattctagcgatgactgcggatgtgatacacgcgacctacgaggaatgtctgaaaagtgggatggatggttacgtctccaaaccttttgaagaagagaatctctataaatccgttgccaaatcattcaaacctaatcctatctcaccttcccggaagagtggaccgattctgctgattgatatgcattttggtttctgtacatacagtaggttcacaatctag

SEQ ID NO: 21 - DNA, 465 bp, Arabidopsis thaliana
atggatttggttcagaagcagaagagtttgcaagattacaccaaatcactcttcttagaagggattttggacagccagttcttgcagctgcaacaactacaagatgaaagcaatccagattttgtttcacaagttgtcacactcttcttccaagactctgataggattctcaatgatctctcactttccctagatcaacaagttgtagactttaaaaaagttgatccccatgttcatcaactcaaaggtagcagctccagtataggagcacagagagttaagaatgcttgtgttgtcttccgcagcttctgcgagcagcaaaatgtcgaagcatgtcatagatgtttgcaacaagtgaagcaagagtattatcttgtgaaaaacagattagagactctgttcaagctggagcaacagattgtagcttctggtggaatgatcccggccgtcgaactcggattttga

SEQ ID NO: 22 - DNA, 817 bp, Arabidopsis thaliana
gaaagacaaaacacaagtttcttcttcttgggattggctatttccagaaatccaagtcaataatcaaagtccaaacaaaaaaatcctctcccaatctccgcttcactcttctcatggacgctctcattgctcagcttcagagacaatttcgtgattacaccatttctctctaccaacaggggtttttggatgatcaatttactgagttgaaaaagctacaagatgatggaagtcctgattttgtgtctgaagtgctttcacttttctttgaagattgtgtgaagcttatcagtaacatggctagagctttggacacgacaggaactgtagattttagtcaggtaggtgctagtgtgcatcaattgaagggtagtagctcaagtgttggtgccaagagggtcaaaactttgtgtgttagcttcaaggaatgttgtgaagctaagaactacgaagggtgtgtgagatgtttgcagcaagtggatattgagtacaaggcgttaaagacaaagcttcaagatatgttcaatcttgagaaacagatcattcaagctggtggtatagttcctcaagtggatattaactaaagagactagtccataagaagaaaaaagatgatgactttctttctttagtttctcttctaaattattttggatttggtgtttgctcaaaaactcaataaaatatgtgcaaaaagaaacaaaaacaagtgatggttgtttataaatcagtagtatgtattgtttgatctcatccgagaaaattgaaaccattggactaatgaatgtgatgataatatatattggtttgcttctcc

SEQ ID NO: 23 - DNA, 510 bp, Arabidopsis thaliana
aactttagctatgaacaccatcgtcgttgctcagttgcagagacaatttcaagactacatcgtttctctttatcaacagggatttctggataatcagttctcagagttgagaaagttgcaagatgaaggaacccctgattttgtagctgaagttgtctctctattcttcgacgactgttccaagcttattaataccatgtctatatccctggagcggccagataatgtggatttcaaacaggtggattcaggtgttcatcaactcaagggtagtagctccagtgtcggtgcaaggagggtgaaaaatgtgtgcatatctttcaaggaatgttgcgatgttcagaaccgtgaagggtgtctaaggtgtttacagcaggtggattatgaatataagatgttaaagactaaacttcaggatctctttaatttagagaaacagatcctccaagctggaggtacaattcctcaagtggatataaattagaccgatgcgtttctcgattatgcaaa

SEQ ID NO: 24 - DNA, 465 bp, Artificial Sequence
  derived from Arabidopsis thaliana
atggatttggttcagaagcagaagagtttgcaagattacaccaaatcactcttcttagaagggattttggacagccagttcttgcagctgcaacaactacaagatgaaagcaatccagattttgtttcacaagttgtcacactcttcttccaagactctgataggattctcaatgatctctcactttccctagatcaacaagttgtagactttaaaaaagttgatccccatgttcaacaactcaaaggtagcagctccagtataggagcacagagagttaagaatgcttgtgttgtcttccgcagcttctgcgagcagcaaaatgtcgaagcatgtcatagatgtttgcaacaagtgaagcaagagtattatcttgtgaaaaacagattagagactctgttcaagctggagcaacagattgtagcttctggtggaatgatcccggccgtcgaactcggattttga

SEQ ID NO: 25 - DNA, 465 bp, Artificial Sequence
  derived from Arabidopsis thaliana
atggatttggttcagaagcagaagagtttgcaagattacaccaaatcactcttcttagaagggattttggacagccagttcttgcagctgcaacaactacaagatgaaagcaatccagattttgtttcacaagttgtcacactcttcttccaagactctgataggattctcaatgatctctcactttccctagatcaacaagttgtagactttaaaaaagttgatccccaagttcaacaactcaaaggtagcagctccagtataggagcacagagagttaagaatgcttgtgttgtcttccgcagcttctgcgagcagcaaaatgtcgaagcatgtcatagatgtttgcaacaagtgaagcaagagtattatcttgtgaaaaacagattagagactctgttcaagctggagcaacagattgtagcttctggtggaatgatcccggccgtcgaactcggattttga

SEQ ID NO: 26 - protein, 1176 aa, Arabidopsis thaliana
MSITCELLNLTSKKAKKSSSSDKKWLKKPLFFLILCGSLVIVLVMFLRLGRSQKEETDSCNGEEKVLYRHQNVTRSEIHDLVSLFSDSDQVTSFECHKESSPGMWTNYGITCSLSVRSDKQETRGLPWNLGLGHSISSTSCMCGNLEPILQQPENLEEENHEEGLEQGLSSYLRNAWWCLILGVLVCHKIYVSHSKARGERKEKVHLQEALAPKKQQQRAQTSSRGAGRWRKNILLLGILGGVSFSVWWFWDTNEEIIMKRRETLANMCDERARVLQDQFNVSLNHVHALSILVSTFHHGKIPSAIDQRTFEEYTERTNFERPLTSGVAYALKVPHSEREKFEKEHGWAIKKMETEDQTVVQDCVPENFDPAPIQDEYAPVIFAQETVSHIVSVDMMSGEEDRENILRARASGKGVLTSPFKLLKSNHLGVVLTFAVYDTSLPPDATEEQRVEATIGYLGASYDMPSLVEKLLHQLASKQTIAVDVYDTTNTSGLIKMYGSEIGDISEQHISSLDFGDPSRNHEMHCRFKHKLPIPWTAITPSILVLVITFLVGYILYEAINRIATVEEDCQKMRELKARAEAADIAKSQFLATVSHEIRTPMNGVLGMLKMLMDTDLDAKQMDYAQTAHGSGKDLTSLINEVLDQAKIESGRLELENVPFDMRFILDNVSSLLSGKANEKGIELAVYVSSQVPDVVVGDPSRFRQIITNLVGNSIKFTQERGHIFISVHLADEVKEPLTIEDAVLKQRLALGCSESGETVSGFPAVNAWGSWKNFKTCYSTESQNSDQIKLLVTVEDTGVGIPVDAQGRIFTPFMQADSSTSRTYGGTGIGLSISKRLVELMQGEMGFVSEPGIGSTFSFTGVFGKAETNTSITKLERFDLAIQEFTGLRALVIDNRNIRAEVTRYELRRLGISADIVSSLRMACTCCISKLENLAMILIDKDAWNKEEFSVLDELFTRSKVTFTRVPKIFLLATSATLTERSEMKSTGLIDEVVIKPLRMSVLICCLQETLVNGKKRQPNRQRRNLGHLLREKQILVVDDNLVNRRVAEGALKKYGAIVTCVESGKAALAMLKPPHNFDACFMDLQMPEMDGFEATRRVRELEREINKKIASGEVSAEMFCKFSSWHVPILAMTADVIQATHEECMKCGMDGYVSKPFEEEVLYTAVARFFEPC

SEQ ID NO: 27 - protein, 1036 aa, Arabidopsis thaliana
MSLFHVLGFGVKIGHLFWMLCCWFVSWFVDNGIEDKSGLLVGSVGDLEKTKMTTLKKKNKMWFWNKISSSGLKIPSFSYQFLGSVKFNKAWWRKLVVVWVVFWVLVSIWTFWYFSSQAMEKRKETLASMCDERARMLQDQFNVSMNHVQAMSILISTFHHGKIPSAIDQRTFSEYTDRTSFERPLTSGVAYAMRVLHSEREEFERQQGWTIRKMYSLEQNPVHKDDYDLEALEPSPVQEEYAPVIFAQDTVSHVVSLDMLSGKEDRENVLRARSSGKGVLTAPFPLIKTNRLGVILTFAVYKRDLPSNATPKERIEATNGYLGGVFDIESLVENLLQQLASKQTILVNVYDITNHSQPISMYGTNVSADGLERVSPLIFGDPLRKHEMRCRFKQKPPWPVLSMVTSFGILVIALLVAHIIHATVSRIHKVEEDCDKMKQLKKKAEAADVAKSQFLATVSHEIRTPMNGVLGMLHMLMDTELDVTQQDYVRTAQASGKALVSLINEVLDQAKIESGKLELEEVRFDLRGILDDVLSLFSSKSQQKGVELAVYISDRVPDMLIGDPGRFRQILTNLMGNSIKFTEKGHIFVTVHLVDELFESIDGETASSPESTLSGLPVADRQRSWENFKAFSSNGHRSFEPSPPDINLIVSVEDTGVGIPVEAQSRIFTPFMQVGPSISRTHGGTGIGLSISKCLVGLMKGEIGFSSTPKVGSTFTFTAVFSNGMQPAERKNDNNQPIFSEFRGMKAVVVDHRPARAKVSWYHFQRLGIRVEVVPRVEQALHYLKIGTTTVNMILIEQEIWNREADDFIKKLQKDPLFLSPKLILLANSVESSISEALCTGIDPPIVIVKPLRASMLAATLQRGLGIGIREPPQHKGPPALILRNLLLGRKILIVDDNNVNLRVAAGALKKYGADVVCAESGIKAISLLKPPHEFDACFMDIQMPEMDGFEATRRIRDMEEEMNKRIKNGEALIVENGNKTSWHLPVLAMTADVIQATHEECLKCGMDGYVSKPFEAEQLYREVSRFFNSPSDTES

SEQ ID NO: 28 - protein, 1057 aa, Arabidopsis thaliana
MNWALNNHQEEEEEPRRIEISDSESLENLKSSDFYQLGGGGALNSSEKPRKIDFWRSGLMGFAKMQQQQQLQHSVAVKMNNNNNNDLMGNKKGSTFIQEHRALLPKALILWIIIVGFISSGIYQWMDDANKIRREEVLVSMCDQRARMLQDQFSVSVNHVHALAILVSTFHYHKNPSAIDQETFAEYTARTAFERPLLSGVAYAEKVVNFEREMFERQHNWVIKTMDRGEPSPVRDEYAPVIFSQDSVSYLESLDMMSGEEDRENILRARETGKAVLTSPFRLLETHHLGVVLTFPVYKSSLPENPTVEERIAATAGYLGGAFDVESLVENLLGQLAGNQAIVVHVYDITNASDPLVMYGNQDEEADRSLSHESKLDFGDPFRKHKMICRYHQKAPIPLNVLTTVPLFFAIGFLVGYILYGAAMHIVKVEDDFHEMQELKVRAEAADVAKSQFLATVSHEIRTPMNGILGMLAMLLDTELSSTQRDYAQTAQVCGKALIALINEVLDRAKIEAGKLELESVPFDIRSILDDVLSLFSEESRNKSIELAVFVSDKVPEIVKGDSGRFRQIIINLVGNSVKFTEKGHIFVKVHLAEQSKDESEPKNALNGGVSEEMIVVSKQSSYNTLSGYEAADGRNSWDSFKHLVSEEQSLSEFDISSNVRLMVSIEDTGIGIPLVAQGRVFMPFMQADSSTSRNYGGTGIGLSISKCLVELMRGQINFISRPHIGSTFWFTAVLEKCDKCSAINHMKKPNVEHLPSTFKGMKAIVVDAKPVRAAVTRYHMKRLGINVDVVTSLKTAVVAAAAFERNGSPLPTKPQLDMILVEKDSWISTEDNDSEIRLLNSRTNGNVHHKSPKLALFATNITNSEFDRAKSAGFADTVIMKPLRASMIGACLQQVLELRKTRQQHPEGSSPATLKSLLTGKKILVVDDNIVNRRVAAGALKKFGAEVVCAESGQVALGLLQIPHTFDACFMDIQMPQMDGFEATRQIRMMEKETKEKTNLEWHLPILAMTADVIHATYEECLKSGMDGYVSKPFEEENLYKSVAKSFKPNPISPSS

SEQ ID NO: 29 - protein, 1057 aa, Artificial Sequence
  derived from Arabidopsis thaliana
MNWALNNHQEEEEEPRRIEISDSESLENLKSSDFYQLGGGGALNSSEKPRKIDFWRSGLMGFAKMQQQQQLQHSVAVKMNNNNNNDLMGNKKGSTFIQEHRALLPKALILWIIIVGFISSGIYQWMDDANKIRREEVLVSMCDQRARMLQDQFSVSVNHVHALAILVSTFHYHKNPSAIDQETFAEYTARTAFERPLLSGVAYAEKVVNFEREMFERQHNWVIKTMDRGEPSPVRDEYAPVIFSQDSVSYLESLDMMSGEEDRENILRARETGKAVLTSPFRLLETHHLGVVLTFPVYKSSLPENPTVEERIAATAGYLGGAFDVESLVENLLGQLAGNQAIVVHVYDITNASDPLVMYGNQDEEADRSLSHESKLDFGDPFRKHKMICRYHQKAPIPLNVLTTVPLFFAIGFLVGYILYGAAMHIVKVEDDFHEMQELKVRAEAADVAKSQFLATVSQEIRTPMNGILGMLAMLLDTELSSTQRDYAQTAQVCGKALIALINEVLDRAKIEAGKLELESVPFDIRSILDDVLSLFSEESRNKSIELAVFVSDKVPEIVKGDSGRFRQIIINLVGNSVKFTEKGHIFVKVHLAEQSKDESEPKNALNGGVSEEMIVVSKQSSYNTLSGYEAADGRNSWDSFKHLVSEEQSLSEFDISSNVRLMVSIEDTGIGIPLVAQGRVFMPFMQADSSTSRNYGGTGIGLSISKCLVELMRGQINFISRPHIGSTFWFTAVLEKCDKCSAINHMKKPNVEHLPSTFKGMKAIVVDAKPVRAAVTRYHMKRLGINVDVVTSLKTAVVAAAAFERNGSPLPTKPQLDMILVEKDSWISTEDNDSEIRLLNSRTNGNVHHKSPKLALFATNITNSEFDRAKSAGFADTVIMKPLRASMIGACLQQVLELRKTRQQHPEGSSPATLKSLLTGKKILVVDDNIVNRRVAAGALKKFGAEVVCAESGQVALGLLQIPHTFDACFMDIQMPQMDGFEATRQIRMMEKETKEKTNLEWHLPILAMTADVIHATYEECLKSGMDGYVSKPFEEENLYKSVAKSFKPNPISPSS

SEQ ID NO: 30 - protein, 1057 aa, Artificial Sequence
  derived from Arabidopsis thaliana
MNWALNNHQEEEEEPRRIEISDSESLENLKSSDFYQLGGGGALNSSEKPRKIDFWRSGLMGFAKMQQQQQLQHSVAVKMNNNNNNDLMGNKKGSTFIQEHRALLPKALILWIIIVGFISSGIYQWMDDANKIRREEVLVSMCDQRARMLQDQFSVSVNHVHALAILVSTFHYHKNPSAIDQETFAEYTARTAFERPLLSGVAYAEKVVNFEREMFERQHNWVIKTMDRGEPSPVRDEYAPVIFSQDSVSYLESLDMMSGEEDRENILRARETGKAVLTSPFRLLETHHLGVVLTFPVYKSSLPENPTVEERIAATAGYLGGAFDVESLVENLLGQLAGNQAIVVHVYDITNASDPLVMYGNQDEEADRSLSHESKLDFGDPFRKHKMICRYHQKAPIPLNVLTTVPLFFAIGFLVGYILYGAAMHIVKVEDDFHEMQELKVRAEAADVAKSQFLATVSHEIRTPMNGILGMLAMLLDTELSSTQRDYAQTAQVCGKALIALINEVLDRAKIEAGKLELESVPFDIRSILDDVLSLFSEESRNKSIELAVFVSDKVPEIVKGDSGRFRQIIINLVGNSVKFTEKGHIFVKVHLAEQSKDESEPKNALNGGVSEEMIVVSKQSSYNTLSGYEAADGRNSWDSFKHLVSEEQSLSEFDISSNVRLMVSIEDTGIGIPLVAQGRVFMPFMQADSSTSRNYGGTGIGLSISKCLVELMRGQINFISRPHIGSTFWFTAVLEKCDKCSAINHMKKPNVEHLPSTFKGMKAIVVDAKPVRAAVTRYHMKRLGINVDVVTSLKTAVVAAAAFERNGSPLPTKPQLDMILVEKDSWISTEDNDSEIRLLNSRTNGNVHHKSPKLALFATNITNSEFDRAKSAGFADTVIMKPLRASMIGACLQQVLELRKTRQQHPEGSSPATLKSLLTGKKILVVDDNIVNRRVAAGALKKFGAEVVCAESGQVALGLLQIPHTFDACFMNIQMPQMDGFEATRQIRMMEKETKEKTNLEWHLPILAMTADVIHATYEECLKSGMDGYVSKPFEEENLYKSVAKSFKPNPISPSS

(19) United States
(12) Patent Application Publication
(10) Pub. No.: US 2006/0185030 A1
(43) Pub. Date: Aug. 17, 2006

(54) Cytokinin response regulators and uses thereof

(76) Inventors: Jen Sheen, Boston, MA (US); Ildoo Hwang, Pohang (KR)

Correspondence Address: CLARK & ELBING LLP, 101 FEDERAL STREET, BOSTON, MA, 02110, US

(21) Appl. No.: 10/479,834

(22) PCT Filed: Jun. 6, 2002
(86) PCT No.: PCT/US02/18066

§ 371 Date: Jul. 16, 2004

Related U.S. Application Data
(60) Provisional application No. 60/296,554, filed on Jun. 6, 2001.

(51) Int. Cl. A01H 1/00; C12N 5/04; C12N 15/82

(52) U.S. Cl. 800/278; 435/419; 435/468

(57) ABSTRACT

The invention generally features methods for increasing yield, shoot formation, and delaying senesence in plants with the use of transgenes that regulate the cytokinin response. The invention also features plants and plant components that harbor the transgene(s).

### BACKGROUND OF THE INVENTION

This invention relates to genetically-engineered plants having increased yield and productivity, shoot, leaf and meristem formation, enhanced photosynthesis, and delayed senescence.

Despite long recognition of cytokinins as essential plant hormones involved in diverse processes of plant growth and development, including cell division, shoot initiation, leaf and root differentiation, chloroplast biogenesis, apical dominance, and senescence, the molecular and biochemical mechanisms underlying cytokinin actions have not been elucidated (Davies, Plant Hormones: Physiology, Biochemistry and Molecular Biology, 1995, Kluwer Academic Publishers, Dordrecht; Meijer and Murray, Curr. Opin. Plant Biol. 4: 44-9, 2001; Mok and Mok, Annu. Rev. Plant Physiol. Plant Mol. Biol. 52: 89-118, 2001; Quilino et al., Trends Plant Sci. 5: 278-82, 2000). Recent genetic identification of Arabidopsis hybrid histidine protein kinases (AHKs), CKI1 and CRE1, in cytokinin signalling (Kakimoto, Science 274: 982-5, 1996; Suzuki et al., Plant Cell. Physiol. 42: 107-13, 2001; Inoue et al., Nature 409: 1060-3, 2001), the characterization of Arabidopsis response regulators (ARRs) as cytokinin primary response genes (D'Agostino et al., Plant Physiol. 124: 1706-17, 2000; Kiba et al., Plant Cell Physiol. 40: 767-71, 1999), and the existence of Arabidopsis histidine-containing phosphotransmitters (AHPs; Suzuki, Imamura, et al., Plant Cell l Physiol. 39: 1258-68, 1998), have implicated the involvement of a two-component phosphorelay mechanism in cytokinin signal transduction. However, cellular and molecular evidence is lacking in supporting a complete cytokinin signalling circuit in plant cells.

Two-component circuitry, consisting of a histidine kinase (HK) sensor and a response regulator (RR) output, are responsible for signal transduction in most prokaryotic and some eukaryotic systems. The signalling pathway is initiated by a HK sensor and mediated by phosphotransfer between a conserved histidine (His) residue in HKs or histidine-containing phosphotransmitters (HPs) and a conserved aspartate (Asp) residue in RRs (Wurgler-Murphy and Saito, Trends Biochein. Sci. 22: 172-6, 1997; Stock et al., Annu. Rev. Biochem. 69: 183-215, 2000). Since there are only one HK, one HP, and two RRs in S. cerevisiae, it was previously believed that two-component signal transduction has limited function in eukaryotes (Stock et al., supra). However, the completion of the Arabidopsis genome has revealed over 40 genes encoding putative two-component signal transducers, AHKs, AHPs and ARRs, suggesting a significant involvement of the ancient and conserved signalling mechanism in many facets of plant cell regulation (Urao, et al., Trends Plant Sci. 5: 67-74, 2000). The identification of conserved HK signature motifs and/or activity in the photoreceptor phytochrome, putative osmosensor, and the ethylene and cytokinin receptors in Arabidopsis further supports this view (Inoue et al., supra; Urao, et al., supra; Yeh and Lagarias, Proc. Natl. Acad. Sci. USA 95: 13976-81, 1998; Bleecker and Kende, Annu. Rev. Cell Dev. Biol. 16: 1-18, 2000).

In eukaryotic signal transduction, the two-component circuit often provides a link between the HK sensor to a MAP kinase (MAPK) signaling cascade. For example, the osmosensing signal transduction pathway in yeast is mediated by the SLN1/PD1/SSK1 phosphorelay. The RR SSK1 then activates the HOG1 MAPK cascade in the cytosol to control gene expression (Maeda et al., Nature 369: 242-5, 1994; Posas et al., Cell 86: 865-75, 1996; Posas and Saito, EMBO J. 17: 1385-94, 1998). It has been speculated that plant HKs such as the ethylene receptor (ETR1) and a putative osmosensor (AHK1) can initiate a phosphorelay and transmit the signal through a MAPK cascade (Bleecker and Kende, supra; Gamble et al., Proc. Natl. Acad. Sci. USA 95: 7825-9, 1998: Clark et al., Proc. Natl. Acad. Sci. USA 95: 5401-6, 1998; Urao et al., Plant Cell 11: 1743-54, 1999). Due to the lack of physiological plant cell assays, the mechanisms of HK action and the signal transduction pathways of any HK-mediated plant responses, including cytokinin signalling, remain obscure.

### SUMMARY OF THE INVENTION

The present invention capitalizes on the discovery that manipulation of the expression of cytokinin response regulators increases plant yield growth, and productivity.

In one aspect, the invention features a method for increasing yield in a plant, the method including the steps of: (a) introducing into plant cells a transgene including DNA encoding a B-type response regulator operably linked to a promoter functional in plant cells to yield transformed plant cells; and (b) regenerating a plant from the transformed cells, wherein the B-type response regulator is expressed in the cells of the transgenic plant, thereby increasing yield in the plant. In preferred embodiments, the B-type response regulator is a crucifer B-type response regulator (for example, ARR1 (SEQ ID NO.: 2); ARR2 (SEQ ID NO.: 3); and-ARR10 (SEQ ID NO.: 9)).

In a second aspect, the invention features a method for increasing yield in a plant, the method including the steps of: (a) introducing into plant cells a transgene operably linked to a promoter functional in plant cells to yield transformed plant cells; and (b) regenerating a plant from the transformed cells, wherein expression of the transgene reduces expression of an A-type response regulator in the cells of the plant, thereby increasing yield in the plant. In preferred embodiments, the A-type response regulator is a crucifer A-type response regulator (for example, ARR 4 (SEQ ID NO.: 5); ARR 5 (SEQ ID NO.: 6); ARR 6 (SEQ ID NO.: 7); and ARR 7 (SEQ ID NO.: 8)). In other preferred embodiments, the transgene expresses an antisense molecule of A-type response regulator; a dominant negative gene product of A-type response regulator; or expression of the transgene results in the co-suppression of the A-type response regulator.

In a third aspect, the invention features a method for increasing shoot formation in a plant, the method including the steps of: (a) introducing into plant cells a transgene including DNA encoding a B-type response regulator operably linked to a promoter functional in plant cells to yield transformed plant cells; and (b) regenerating a plant from the transformed cells, wherein the B-type response regulator is expressed in the cells of the plant, thereby increasing shoot formation in the plant. In preferred embodiments, the method includes the use of a B-type response regulator that is a crucifer B-type response regulator.

In a fourth aspect, the invention features a method for increasing shoot formation in a plant, the method including the steps of: (a) introducing into plant cells a transgene operably linked to a promoter functional in plant cells to yield transformed plant cells; and (b) regenerating a plant from the transformed cells, wherein expression of the transgene reduces expression of an A-type response regulator in the cells of the plant, thereby increasing shoot formation in the plant.

In a fifth aspect, the invention features a method for delaying senescence in a plant, the method including the steps of: (a) introducing into plant cells a transgene including DNA encoding a B-type response regulator operably linked to a promoter functional in plant cells to yield transformed plant cells; and (b) regenerating a plant from the transformed cells, wherein the B-type response regulator is expressed in the cells of the plant, thereby delaying senescence in the plant.

In a sixth aspect, a method for delaying senescence in a plant, the method including the steps of: (a) introducing into plant cells a transgene operably linked to a promoter functional in plant cells to yield transformed plant cells; and (b) regenerating a plant from the transformed cells, wherein expression of the transgene reduces expression of an A-type response regulator in the cells of the plant, thereby delaying senescence in the plant.

In a seventh aspect, the invention features a method for increasing yield in a plant, the method including the steps of: (a) introducing into plant cells a transgene including DNA encoding a histidine kinase operably linked to a promoter functional in plant cells to yield transformed plant cells; and (b) regenerating a plant from the transformed cells, wherein histidine kinase is expressed in the cells of the plant, thereby increasing yield in the plant. In preferred embodiments, the histidine kinase is a crucifer histidine kinase (for example, CKI1(SEQ ID NO.: 13) and CRE1 (SEQ ID NO.: 18)).

In an eighth aspect, the invention features a method for increasing shoot formation in a plant, the method including the steps of: (a) introducing into plant cells a transgene including DNA encoding a histidine kinase operably linked to a promoter functional in plant cells to yield transformed plant cells; and (b) regenerating a plant from the transformed cells, wherein histidine kinase is expressed in the cells of the plant, thereby increasing shoot formation in the plant.

In a ninth aspect, a method for delaying senescence in a plant, the method including the steps of: (a) introducing into plant cells a transgene including DNA encoding a histidine kinase operably linked to a promoter functional in plant cells to yield transformed plant cells; and (b) regenerating a plant from the transformed cells, wherein histidine kinase is expressed in the cells of the plant, thereby delaying senescence in the plant.

The invention further includes plants and plant components expressing one, or a combination of two or more, of the aforementioned transgenes. The invention also encompasses methods of generating these plants or plant components, which may involve introducing the transgenes individually to separate plants or plant components, and then crossing the appropriate genotypes to get the desired transgene combination. Alternatively, one or more constructs expressing a desired combination of transgenes may be generated and then introduced into the plant or plant component.

For example, the invention also relates to a plant or plant component comprising at least one transgene encoding (i) an A-type response regulator polypeptide, (ii) an antisense A-type response regulator RNA, (iii) a dominant negative A-type response regulator polypeptide, (iv) a B-type response regulator polypeptide, (v) an antisense B-type response regulator RNA, (vi) a dominant negative B-type response regulator, (vii) an antisense HK RNA, (viii) a dominant negative HK polypeptide, or any combination of (i) lviii). In addition, the invention includes any of the aforementioned plants further including a transgene encoding a histidine kinase polypeptide.

The invention further includes a plant or plant component that includes: (a) a first transgene encoding (i) an A-type response regulator polypeptide, (ii) an antisense A-type response regulator RNA, or (iii) a dominant negative A-type response regulator polypeptide or combination thereof; (b) a second transgene encoding (iv) a B-type response regulator polypeptide, (v) an antisense B-type response regulator RNA, (vi) a dominant negative B-type response regulator or combination thereof, and (c) a third transgene encoding (vii) a HK polypeptide; (viii) an antisense HK RNA, (ix) a dominant negative HK polypeptide or combination thereof.

Exemplary plants which are useful in the methods of the invention, as well as for generating the plants (or plant cells, plant components, plant tissues, or plant organs) of the invention, include dicots and monocots, such as sugar cane, wheat, rice, maize, sugar beet, barley, manioc, crucifer, mustard, potato, soybean, sorghum, cassava, banana, grape, oats, tomato, millet, coconut, orange, rye, cabbage, apple, eggplant, watermelon, canola, cotton, carrot, garlic, onion, pepper, strawberry, yam, papaya, peanut, onion, legume, bean, pea, mango, and sunflower.

By “operably linked” is meant that a gene and a regulatory sequence(s) are connected in such a way as to permit gene expression when the appropriate molecules (for example, transcriptional activator proteins) are bound to the regulatory sequence(s).

By “plant cell” is meant any self-propagating cell bounded by a semi-plant component expression control permeable membrane and containing a plastid. Such a cell also requires a cell wall if further propagation is desired. Plant cell, as used herein, includes, without limitation, algae, cyanobacteria, seeds, suspension cultures, embryos, meristematic regions, callus tissue, leaves, roots, shoots, gametophytes, sporophytes, pollen, and microspores.

By “plant component” is meant a part, segment, or organ obtained from an intact plant or plant cell. Exemplary plant components include, without limitation, somatic embryos, leaves, stems, roots, flowers, tendrils, fruits, scions, and rootstocks.

By “transgene” is meant any piece of DNA which is inserted by artifice into a cell, and becomes part of the genome of the organism which develops from that cell. Such a transgene may include a gene which is partly or entirely heterologous (i.e., foreign) to the transgenic organism, or may represent a gene homologous to an endogenous gene of the organism.

By “transgenic” is meant any cell which includes a nucleic acid sequence (e.g., a recombinant DNA sequence) which is inserted by artifice into a cell and becomes part of the genome of the organism which develops from that cell. As used herein, the transgenic organisms are generally transgenic plants and the DNA (transgene) is inserted by artifice into the nuclear or plastidic genome.

By “yield” or “plant yield” is meant increased growth (e.g., crop growth) or increased biomass. For example, increased yield results from increased shoot growth or meristem formation. Plants expressing the genes disclosed herein exhibiting increased yield can be selected by visual observation, for example by comparison with a wild-type plant.

By “reducing expression” or “reduces expression” is meant a decrease in the level of gene expression (for example, expression of a gene encoding a A-type response regulator) by at least 30-50%, preferably by at least 50-80%, and more preferably by at least 80-95% or greater relative to the level in a control plant (for example, a wild-type plant). Reduction of such expression levels may be accomplished by employing standard methods which are known in the art including, without limitation, antisense and co-suppression technologies, expression of a dominant negative gene product, or through the generation of mutated genes using standard mutagenesis techniques. Levels of negative regulator polypeptide or transcript are monitored according to any standard technique including, but not limited to, northern blotting, RNase protection, or immunoblotting.

By “crucifer” is meant any plant that is classified within the Cruciferae family. The Cruciferae include many agricultural crops, including, without limitation, rape (for example, Brassica campestris and Brassica napus), broccoli, cabbage, brussel sprouts, radish, kale, Chinese kale, kohlrabi, cauliflower, turnip, rutabaga, mustard, horseradish, and Arabidopsis.

By “a promoter functional in a plant cell” is meant any minimal sequence sufficient to direct transcription in a plant cell. Included in the invention are promoter elements that are sufficient to render promoter-dependent gene expression controllable for cell-, tissue-, or organ-specific gene expression, or elements that are inducible by external or internal agents (for example, cytokinins), or elements that are capable of cycling gene transcription; such elements may be located in the 5′ or 3′ regions of the native gene or engineered into a transgene construct.

The invention provides a number of important advances and advantages for improving and enhancing agronomically important traits such as photosynthesis, productivity, yield, leaf, shoot, and meristem formation, and delaying senescence. In particular, the invention provides for increased production efficiency, as well as for improvements in quality and yield of crop plants and ornamentals. Thus, the invention contributes to the production of high quality and high yield agricultural products; for example, fruits, ornamentals, vegetables, cereals, and field crops. Genetically-improved seeds and other plant products that are produced using plants expressing the genes and methods described herein also render farming possible in areas previously unsuitable for agricultural production The mechanisms disclosed herein for increasing plant yield and productivity is expected to be ubiquitous throughout the plant kingdom.

Other features and advantages of the invention will be apparent from the following description of the preferred embodiments thereof, and from the claims.

### BRIEF DESCRIPTION OF THE DRAWING

FIG. 1A is a bar graph showing the specificity of plant hormone responses in the Arabidopsis mesophyll protoplast transient expression system.

FIG. 1B is a graph depicting a cytokinin dose response for the ARR6 promoter induction.

FIG. 1C is a bar graph showing the induction of the ARR6 promoter by different cytokinins.

FIG. 1D shows that CKI1 activation of ARR6-LUC requires HK activity and phosphorelay. The photograph (top) shows an autoradiogram depicting the expression of CKI1 proteins. The bar graph (bottom) shows the results of transfected protoplasts that were incubated for 3 hours to allow effector gene expression before cytokinin treatment for another 3 hours.

FIG. 1E shows that CRE1 confers cytokinin hypersensitivity on the activation of the ARR6-LUC reporter. The photograph (top) shows an autoradiogram depicting the expression of Arabidopsis HK proteins. The results of this incubation with cytokinin are shown in the bar graph (bottom).

FIG. 1F shows a series of a photomicrographs showing that CKI1 is localized at the plasma membrane.

FIG. 2A demonstrates that AHP acts as a shuttle between the cytosol and nucleus in cytokinin signalling. The photograph (top) depicts an autoradiogram demonstrating expression of AHP proteins. The bar graph (bottom) shows the effect of cytokinin treatment on plasmid expression.

FIG. 2B shows a series of photomicrographs demonstrating that cytokinin induces transient AHP translocation.

FIG. 3A shows negative and positive regulation by ARRs. The photograph depicts an autoradiogram (top) showing the expression of Arabidopsis ARR proteins. The bar graph (bottom) shows the activity of ARR6 promoter activity when cells were then incubated with or without 100 nM t-zeatin for another 3 hours before the activity was measured.

FIG. 3B shows that ARRs act downstream of CKI1 in cytokinin signalling.

FIG. 3C shows a series of photomicrographs demonstrating that Arabidopsis ARRs are localized in the nucleus.

FIG. 4A is a photograph showing that ectopic expression of ARR2 is sufficient to promote cytokinin responses in transgenic tissues and plants when exposed to auxin.

FIG. 4B is a photograph showing that ectopic expression of ARR2 is sufficient to promote cytokinin responses in transgenic tissues and plants when exposed to both auxin and cytokinin.

FIG. 4C is a photograph demonstrating that cytokinin signaling delays dark-induced senescence in transgenic Arabidopsis plants.

FIG. 5 is a schematic of a model of the cytokinin signal transduction pathway in Arabidopsis.

### DETAILED DESCRIPTION

Overview

Cytokinins are essential plant hormones involved in shoot meristem and leaf formation, cell division, chloroplast biogenesis, and senescence. Although hybrid histidine protein kinases (HKs) have been implicated in cytokinin perception in Arabidopsis, the action of HK receptors and downstream signalling pathways remain elusive in plant cells. To study the action of HK receptors and downstream signaling pathways, a mesophyll protoplast transient assay based on the transcription activation of a cytokinin primary response gene ARR6 in Arabidopsis was developed. The use of this high-throughput cell assay for functional genomic analysis of the two-component regulators has provided a powerful tool to decipher the first complete cytokinin signalling pathway. Rather than following the established eukaryotic HK and MAPK cascade paradigm, plant two-component signalling can integrate multiple HK activities to common AHPs as cytoplasm and nuclear shuttles, and distinct nuclear ARRs as master regulatory outputs.

As is described in more detail below, a new eukaryotic two-component signalling circuit that initiates cytokinin signalling by employing distinct hybrid HK activities at the plasma membrane has been identified. In particular, AHPs are shown to act as novel eukaryotic signalling shuttles between the cytoplasm and nucleus in a transient and cytokinin-dependent manner. The short signalling circuit reaches the nuclear target genes by de-repressing nuclear response regulators ARR1, ARR2, and ARR10 as transcription activators. The cytokinin-inducible ARR4, ARR5, ARR6, and ARR7 act as transcription repressors, suggesting a negative feedback loop. Thus, the Arabidopsis cytokinin signal transduction pathway consists of four major steps: HK sensing and signalling, AHP translocation, ARR de-repression, and a negative feedback loop by cytokinin-inducible ARRs. Transgenic tissue and plant analyses support the importance of this core signalling pathway in diverse cytokinin responses. Furthermore, ectopic expression of the master regulator ARR2 as the rate-limiting two-component output in transgenic Arabidopsis was sufficient to mimic cytokinin in promoting shoot meristem proliferation, leaf differentiation, expansion, and longevity.

The examples provided below are for the purposes of illustrating the invention, and should not be construed as limiting.

Cytokinin Signal is Perceived and Initiated by the Multiple HK Receptors

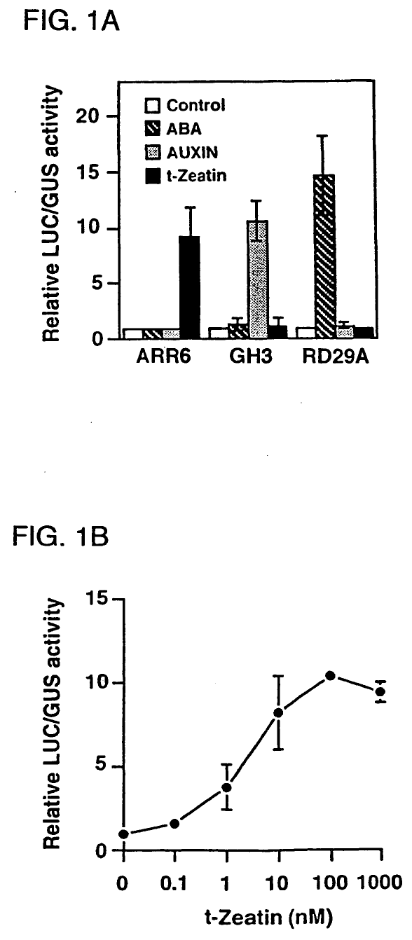

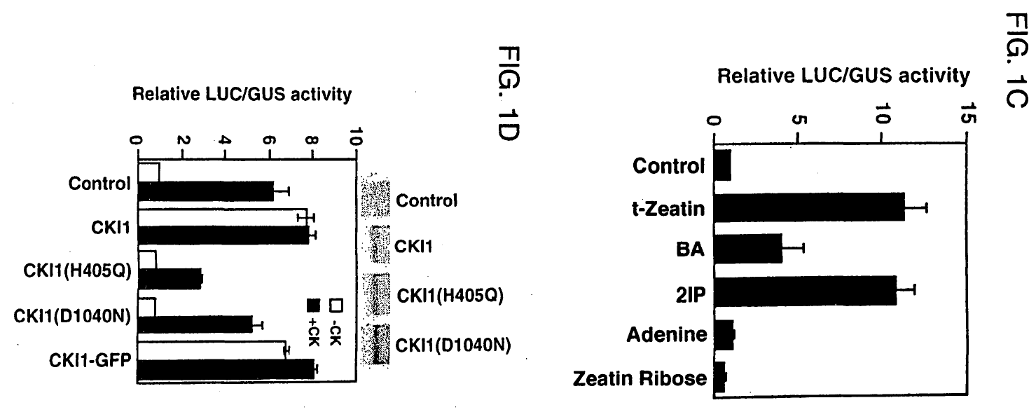

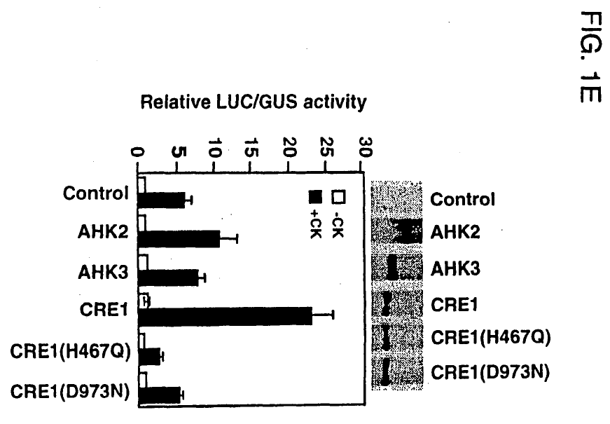

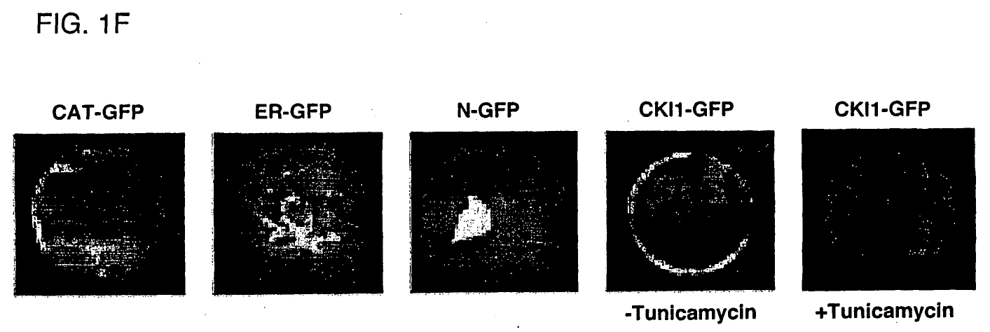

To elucidate the regulatory circuitry in cytokinin signal transduction, a leaf cell assay based on cytokinin inducible transcription in Arabidopsis mesophyll protoplasts was developed. The 2.4 kb promoter of an Arabidopsis cytokinin primary response gene (SEQ ID NO.: 1), encoding the response regulator 6 (ARR6), was fused to the firefly luciferase (LUC) reporter. In FIG. 1A, transfected protoplasts were incubated with or without 100 nM t-zeatin, 1 μM indole-3-acetic acid (IAA), or 100 μM ABA. After 3 hours of incubation, LUC and GUS activities were determined. In transfected protoplasts, the activity of ARR6-LUC was specifically induced by cytokinin but not by other plant hormones such as abscisic acid (ABA) or auxin (FIG. 1A).

In the same system, the GH3 promoter was only activated by auxin, whereas the RD29A promoter was solely induced by abscisic acid (ABA), demonstrating the specificity of three plant hormone responses in Arabidopsis mesophyll protoplasts (FIG. 1A). The activity of ARR6-LUC was induced by physiological concentration of a natural cytokinin trans-zeatin (t-zeatin) from 1 to 100 nM (FIG. 1B).

To further show the specificity of the cytokinin response, various active and inactive cytokinin analogues were examined. Only the active cytokinins, t-zeatin, 2-isopentenyladenine (2-IP; 100 nM) and benzyladenine (BA; 100 nM), induced the reporter gene ARR6-LUC (FIG. 1C).

The response in the protoplast system is similar to the cytokinin activation of various ARR genes shown in planta (D'Agostino et al., supra; Kiba et al., supra). Thus, we have established a reliable and physiological system to dissect the regulatory components in the cytokinin signal transduction pathway in Arabidopsis.

Cytokinin Signalling by Distinct HK Receptors and Phosphorelay

Despite the genetic identification of histidine kinases (HKs) as receptors for the plant hormones ethylene and cytokinin in Arabidopsis, whether or where HK activity is important for signal transduction in plant cells remains an unresolved question due to the lack of physiological plant cell assays. CKI1, a hybrid HK with a conserved response regulator (RR) receiver domain has been implicated in cytokinin responses (Kakimoto, supra). The enhanced CKI1 expression in Arabidopsis shooty callus mutants also supports its role in cytokinin signalling (Frank et al., Plant Physiol. 122: 721-9, 2000). However, the biochemical mechanism of CKI1 function and its role in cytokinin signalling are still unknown. To determine whether CKI1 mediates cytokinin responsive transcription, the full-length CKI1 gene (SEQ ID NO.: 13) was tagged with double haemagglutinin epitope (DHA) sequence and fused to a constitutive promoter, and co-transfected with ARR6-LUC into the Arabidopsis protoplasts. Specifically, protoplasts were cotransfected with the ARR6-LUC reporter and the effector plasmid expressing CKI1 (SEQ ID NO.: 13), CKI1(H405Q) (SEQ ID NO.: 14), CKI1(D1050N) (SEQ ID NO.: 15), CKI1-GFP, or mER7-GFP (control). As shown in FIG. 1D, the wild-type CKI1 activated the ARR6 promoter without exogenous t-zeatin. It is possible that over-expression of CKI1 renders protoplasts hypersensitive to endogenous cytokinin and/or exceeds the capacity of negative regulators. Alternatively, CKI1 could encode a constitutively active HK connected to the cytokinin signal transduction pathway. Cytokinin treatment hardly enhanced the reporter gene activity in the presence of CKI1 (FIG. 1D).

To determine whether the HK activity and phosphorelay are required for CKI1 activation of the ARR6 promoter, the conserved His 405 and Asp 1040 residues in the HK and phosphorylation sites, respectively, were mutated. Specifically, protoplasts were transfected with plasmid DNA expressing CKI1 (SEQ ID NO.: 13), CKI (H405Q) (SEQ ID NO.: 14) and CKI1(D1040N) (SEQ ID NO.: 15), or vector DNA as a control. The [³⁵S]methionine labeled protein from transfected protoplasts was irnmunoprecipitated with anti-HA and protein A-agarose, and analyzed by SDS-PAGE. Despite comparable expression of the CKI1(H405Q) and CKI1(D1050N) mutants as the wild-type CKI1, neither could activate the expression of ARR6-LUC (FIG. 1D). Furthermore, the CKI1(H405Q) mutant served as a strong dominant negative mutant to block the activation of the ARR6 promoter by exogenous t-zeatin. The CKI1 (D1040N) mutant is less potent. These results indicated that HK activity and phosphorelay from His 405 to D1040 are required for the CKI1 function in activating cytokinin signalling. Moreover, the dominant negative CKI1(H405Q) mutant might interfere with cytokinin perception and/or disturb downstream signalling.

Recently, another Arabidopsis hybrid HK, CRE1/AHK4/WOL, has been shown to be a cytokinin receptor. The evidence is based on the inability of the cre1 mutant to respond to cytokinin in the shoot induction assay and its ability to complement HK mutants of budding and fission yeast and E. coli in a cytokinin-dependent manner (Suzuki et al., supra; Inoue et al., supra; Mahonren et al., Genes Dev. 14: 2938-43, 2000). Since CRE1/AHK4/WOL is predominantly expressed in roots (Mahonen et al., supra; Ueguchi et al., Plant Cell Physiol. 42: 231-5, 2001) and the cre1 and wol recessive mutants lack an obvious leaf phenotype (Inoue et al., supra; Mahonen et al., supra; Ueguchi et al., supra), it is possible that other closely related Arabidopsis HK genes, such as AHK2 and AHK3, and CKI1 provide cytokinin receptor functions in promoting shoot meristem and leaf formation, and delaying leaf senescence (Inoue et al., supra; Ueguchi et al., supra).

To directly test the function of these HKs in cytokinin signalling, constructs expressing AHK2 (SEQ ID NO.: 16), AHK3 (SEQ ID NO.: 17), or CRE1(AHK4/WOL) (SEQ ID NO.: 1S) were cotransfected into Arabidopsis protoplast with the cytokinin-inducible reporter ARR6-LUC. Specifically, protoplasts were cotransfected with the ARR6-LUC reporter and the effector plasmid expressing AHK2, AHK3, CRE1(AHK4/WOL), CRE1(H467Q) (SEQ ID NO.: 19), CRE1(D973N) (SEQ ID NO.: 20), or mER7-GFP (control), and were analyzed after 6 hours of cytokinin treatment. Different from the CKI1 activation, over-expression of AHK2, AHK3, or CRE1(AHK4/WOL) did not enhance the expression of ARR6-LUC from the basal level in the absence of exogenous cytokinin. However, cytokinin treatment resulted in further activation of the reporter, especially for CRE1(AHK4/WOL) (FIG. 1E). The requirement of the conserved His 467 and Asp 973 residues in the HK and the receiver domain, respectively, for the CRE1 activity was also tested. In FIG. 1E top, the expression of Arabidopsis HK proteins was detected by [³⁵S]methionine-labeled AHK2 (SEQ ID NO.: 26), AHK3 (SEQ ID NO.: 27), CRE1(AHK4/WOL) (SEQ ID NO.: 28), CRE1(H467Q) (SEQ ID NO.: 29), and CRE1(D973N) (SEQ ID NO.: 30) that were immunoprecipitated and analyzed by SDS-PAGE. CRE1(H467Q) and CRE1(D973N) mutants lost their ability to further enhance ARR6-LUC expression in the presence of exogenous cytokinin (FIG. 1E).

Moreover, both mutants also imposed a dominant negative effect on blocking cytokinin signalling. Similar to the CKI1 mutants, the His mutant is more potent than the Asp mutant, perhaps due to the flexible phosphorelay process between mutant and wild-type HKs. These results indicated that cytokinin signals are sensed by multiple HK receptors with different or overlapping expression patterns. Although CRE1 is predominantly expressed in roots (Mahonen et al., supra; Ueguchi et al., supra), it can certainly function in leaf cells as a cytokinin receptor. However, CKI1 and CRE1 represent two different types of cytokinin receptors that require HK activity and phosphorelay to initiate cytokinin signalling, but have different regulatory mechanisms and probably distinct affinity or specificity for cytokinins.

To gain insight into where the cytokinin signalling is perceived, the sub-cellular localization of CKI1 was examined using the CKI1-GFP fusion. We first confirmed that CKI1-GFP acted similarly as CKI1-HA in the protoplast assay based on ARR6-LUC activation (FIG. 1D). Confocal microscopy showed that CKI1 is mainly localized to the plasma membrane but not in the cytosol, ER or nucleus, indicated by various GFP markers (FIG. 1F). Specifically, protoplasts were transfected with CKI1-GFP or various GFP marker plasmid DNA. Tunicamycin (1 μg/ml) treatment was for 12 hours. The transfected protoplasts were analyzed with a confocal microscope. The subcellular GFP markers are “CAT-GFP” for the cytosol, “ER-GFP” for the endoplasmic reticulum, and “N-GFP” (HSP-GFP) for the nucleus. Since the N-terminus of CKI1 is replete with putative glycosylation sites, the effect of a glycosylation inhibitor, tunicamycin, in CKI1-GFP expression, localization or function was examined. Although tunicamycin did not affect the expression of other GFP constructs, the expression of CKI1-GFP was completely abolished (FIG. 1F). This result showed that the glycosylation of CKI1 is important for its stability, processing and/or trafficking in plant cells.

HPs Acts as a Shuttle Between the Cytosol and Nucleus Shuttles in Cytokinin Signalling

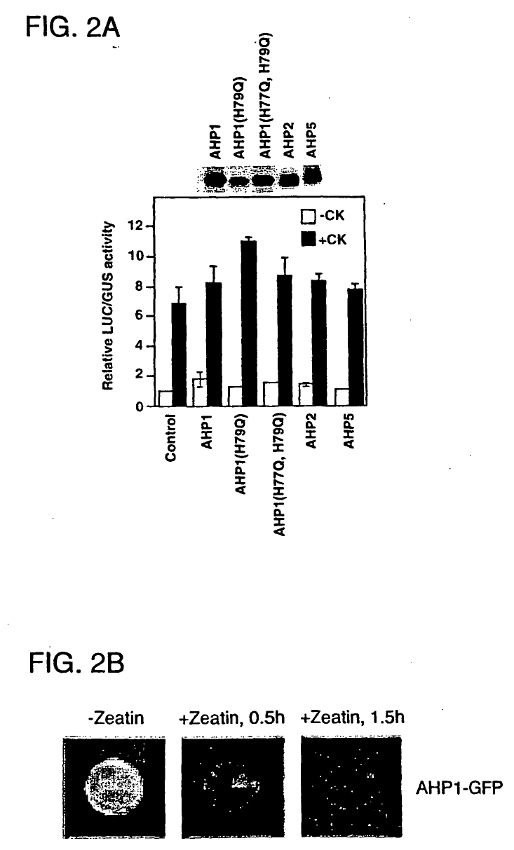

The studies of multistep phosphorelay in yeast osmosensing and in various prokaryotic systems have shown the use of histidine-containing phosphotransmitters (HP) as signal transducers between receptors and response regulators (Stock et al., supra; Posas et al., supra; Uhl and Miller, EMBO J. 15: 1028-36, 1996). The analysis of the Arabidopsis genome has revealed at least five genes encoding putative AHPs with unknown physiological functions. To determine whether AHPs are involved in the cytokinin-signal transduction, AHP1, AHP2, and AHP5 were cloned into the plant expression vector and tested in the protoplast cytokinin response assay based on the ARR6-LUC reporter activity. AHP1, AHP2, and AHP5 slightly increased the expression of the ARR6-LUC with or without t-zeatin (FIG. 2A). Specifically, protoplasts were cotransfected with the ARR6-LUC reporter and an effector plasmid carrying AHP1 (SEQ ID NO.: 21), AHP2 (SEQ ID NO.: 22), AHP5 (SEQ ID NO.: 23), or AHP1 mutants (SEQ ID NOS.: 24 and 25). Transfected protoplasts were incubated for 6 hours to allow effector expression before cytokinin treatment for 3 hours. The AHP1 mutant, where the conserved His79 was replaced with Gln, did not repress the cytokinin activation of ARR6-LUC. Even the AHP1(H77Q, H79Q; SEQ ID NO.: 25) double mutant was not able to inhibit the ARR6-LUC activation by cytokinin. AHPs and AHP1 mutants were expressed at comparable levels in the protoplast system (FIG. 2A top). This result indicated that AHPs are not the limiting factor or the key switch/regulators in cytokinin signalling. Their action as potential mediators may require cytokinin-dependent modification. Previous in vitro studies have suggested that AHP1 and AHP2 could interact with the receiver domain of CKI1 (Nakamura et al., Biosci. Biotechitol. Biochein. 63: 1627-30, 1999).

To gain further in vivo support for the involvement of AHPs in cytokinin signalling, the action of AHP1-GFP in transfected protoplast in the absence or presence of cytokinin was followed using fluorescence microscopy. Specifically, protoplasts were transfected with AHP1-GFP, AHP2-GFP, or AHP5-GFP and incubated for 3 hours to allow sufficient expression. After t-zeatin (100 nM) treatment for 30 minutes, transfected cells were observed under a fluorescence microscope. As shown in FIG. 2B, AHP1-GFP was mainly localized in the cytoplasm without cytokinin stimulation, but was translocated into the nucleus by t-zeatin treatment. The cytokinin-dependent translocation of AHP1-GFP was transient, occurring within 30 to 90 minutes after cytokinin treatment (FIG. 2B). AHP2-GFP showed similar cytokinin-dependent translocation but not AHP5-GFP, suggesting their different functions in plant cells. Thus, AHP1 and AHP2 are believed to serve as shuttles and phosphorelay carriers between the cytokinin receptors and the downstream nuclear responses.

Opposite Regulation of Early Cytokinin Response Gene Transcription by Two Types of Nuclear RRs in Cytokinin Signalling

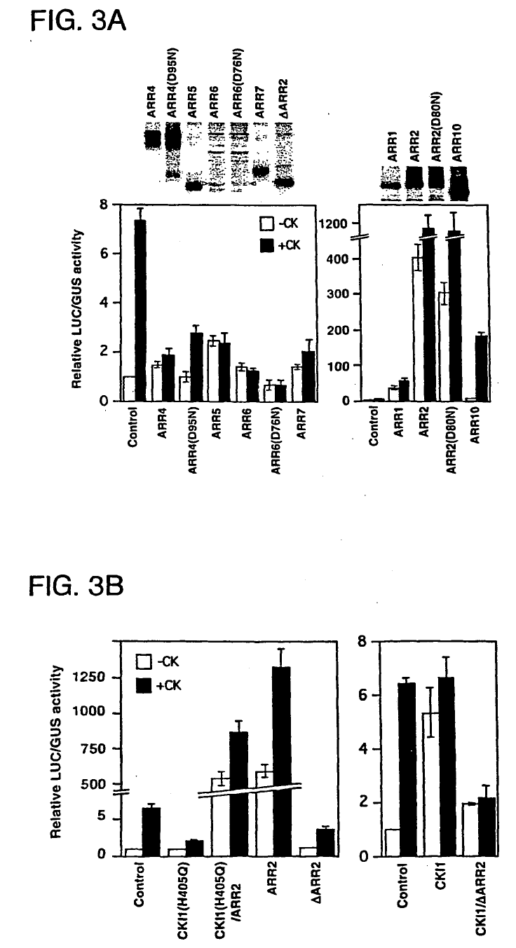

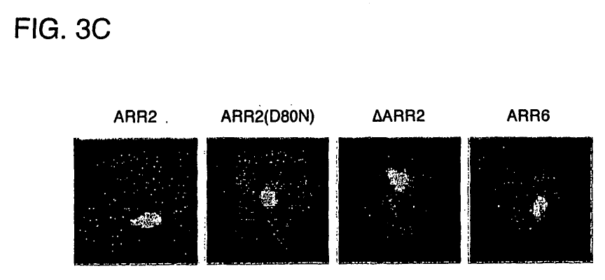

The requirement of HK activity and phosphorelay in the initiation of cytokinin signalling from CKI1 and CRE1, the cytokinin-inducible ARR6 expression, the cytokinin-dependent translocation of AHPs into the nucleus, and the physical interaction of AHPs and ARRs in yeast (Suzuki et al., Plant Cell Physiol. 42: 37-45, 2001) suggest that some ARRs may act downstream in cytokinin signal transduction. There are two major subfamilies of RR genes in the Arabidopsis genome: the cytokinin-inducible A type and the DNA-binding B type (Urao, et al., supra; D'Agostino and Kieber, Trends Biochem. Sci. 24: 452-6, 1999; Sakai et al., Plant J. 24: 703-11, 2000). To investigate whether any Arabidopsis RRs are involved in cytokinin signalling, four representative A-type ARR genes and three B-type ARR genes were cloned into a standard plant expression vector with an HA or GFP tag. Specifically, protoplasts were cotransfected with the ARR6-LUC reporter and the effector plasmid carrying ARR1, ARR2, ARR4, ARR5, ARR6, ARR7, ARR10, or various mutants, ARR2 (D80N) (SEQ ID NO.: 10), ARR4(D95N) (SEQ ID NO.: 11), and ARR6(D76N) (SEQ ID NO.: 12). As shown in FIG. 3A, A-type ARRs, such as ARR4, ARR5, ARR6, and ARR7, repressed ARR6-LUC activity induced by 100 nM t-zeatin. Although ARR6 seemed to be expressed at lower abundance, its repression activity was the strongest.

In contrast, the B-type ARRs, such as ARR1, ARR2, and ARR10, dramatically activated ARR6-LUC expression. Over-expression of ARR1, ARR2, and ARR10 was sufficient to activate cytokinin signalling at different levels in the absence of exogenous cytokinin. ARR1 and ARR2 activated ARR6-LUC about 40- and 400-fold, respectively. Cytokinin treatment further enhanced the effect of ARR2 on ARR6-LUC to over 1000-fold. The lower activation by ARR1 could be due to its lower expression level in transfected protoplasts. In the absence of cytokinin, ARR10 could activate ARR6-LUC about 10 fold. Cytokinin enhanced the effect of ARR10 on the ARR6-LUC activity another 10-20 fold. The differential effect of ARR2 and ARR10 in activating cytokinin signalling could be attributed to differences in their intrinsic activities in DNA binding and/or transcription activation. It could also be due to their distinct affinity to endogenous repressors that likely exist in leaf protoplasts to prevent cytokinin signalling under unstimulated condition. Surprisingly, mutations of the conserved Asp residue in the receiver domains of ARR4(D95N), ARR6(D76N), and ARR2(D80N) did not lead to alteration of their repression and activation functions, respectively, in cytokinin signalling (FIG. 3A). The transfected cells were incubated for 3 hours to allow effector protein expression. This result is consistent with the model that phosphorelay stimulated by cytokinin may be involved in de-repression of the positive regulators like ARR1, ARR2, and ARR10. Over-expression of these ARRs likely bypasses the negative regulation and causes constitutive cytokinin responses without the signal.

To further support the role of ARR2 in cytokinin signalling, we designed a dominant mutant of ARR2 (ΔARR2) (SEQ ID NO.: 4) containing only the DNA binding domain but not the putative transcription activation and the receiver domains (Sakai et al., supra). If ΔARR2 can compete with the endogenous ARRs in binding to the ARR6 promoter without transcription activation, it may block cytokinin- or CKI1-dependent activation of ARR6-LUC. In FIG. 3B, protoplasts were cotransfected with the ARR6-LUC reporter alone or with the effector plasmids as indicated. Transfected protoplasts were incubated for 6 hours to allow effector protein expression before treatment with 100 nM t-zeatin for 3 hours. As shown in FIG. 3B, the dominant negative mutant of ARR2 could effectively block ARR6-LUC expression in the presence of 100 nM t-zeatin or CKI1, suggesting that ARR2 and/or ARR2-like proteins act downstream of cytokinin and CKI1-initiated phosphorelay as the nuclear targets in the cytokinin signal transduction pathway. Furthermore, the dominant negative effect of the CKI1 (H450Q) mutant on the protoplast response to exogenous cytokinin could be bypassed by over-expression of the wild-type ARR2 (FIG. 3B). These epistasis analyses place ARR2 downstream of multiple cytokinin HK receptors. The finding of multiple ARR2 binding motifs, (G/A)GAT(T/C), in the promoter regions of ARR6 and other cytokinin-inducible genes suggests that ARR2 could be a master regulator in cytokinin signalling (Id.). Exogenous cytokinin-initiated phosphorelay through CRE1, or AHK2 and AHK3 likely converges with the CKI1 phosphorelay on the common AHP shuttles and the nuclear ARR transcription activators.

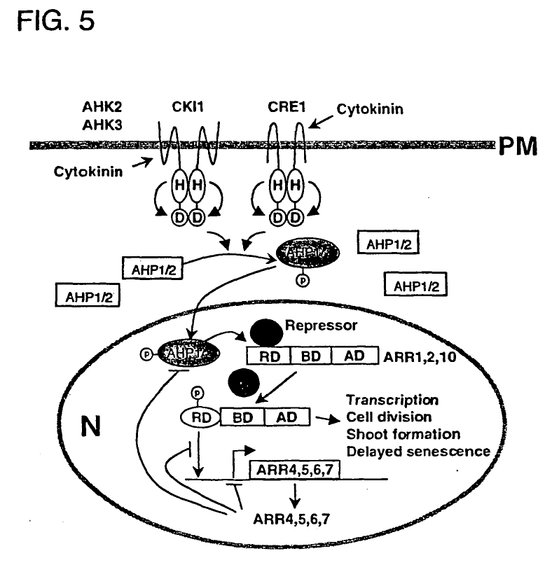

To provide more evidence that the A-type and B-type ARRs are transcription repressors and activators, respectively, in cytokinin signalling, the sub-cellular localization of ARR6-GFP and ARR2-GFP was examined. In FIG. 3C, Protoplasts were transfected with ARR2-GFP, ARR2(D80N)-GFP, ΔARR2-GFP, or ARR6-GFP plasmid DNA and observed after 3-6 hours of incubation with a fluorescence microscope. Both types of ARRs are exclusively localized in the nucleus regardless of the cytokinin treatment (FIG. 3C). There nuclear localization is likely independent of the phosphorylation state because ARR2(D80N)-GFP was found in the nucleus. The ARR2 dominant negative mutant (ΔARR2) was also localized in the nucleus (FIG. 3C). Thus, the cytokinin-dependent phosphorelay does not play a role in the nuclear localization and DNA-binding of ARRs or their intrinsic transcription activation or repression activities. The results support the model that cytokinin-initiated phosphorelay plays a role in releasing the sequestered activation-type ARR transcription factors from the yet unknown repressors (FIG. 5).

As depicted in FIG. 5, cytokinin signal is perceived internally or externally by multiple HKs at the plasma membrane (AHK2, AHK3, CKI1, or CRE1). Upon perception of the cytokinin signal, HKs initiate a signalling cascade via the phosphorelay that in turn results in the nuclear translocation of AHPs from the cytosol. Activated AHPs interact with sequestered ARRs or ARR complex and release the activation type of ARRs from putative repressor in the nucleus. The de-repressed ARRs bind to multiple cis elements in the promoter of target genes. The transcription activation is essential for cell proliferation, shoot formation, and delayed senescence. The activation of the repressor-type of ARRs as early cytokinin response genes provides a negative feedback mechanism that allows plant cells for fine-tuning or resetting the physiological state in diverse differentiation processes during plant growth and development (RD, response domain; BD, DNA binding domain; AD, transcription activation domain; PM, plasma membrane; N, nucleus; R, repressor).

This model is consistent with the findings that the N-terminus of ARR1 or ARR2 inhibits transcription based on a synthetic promoter (Id.), and the addition of cytokinin further enhances the effects of ARR1, ARR2 and ARR10 over-expression on the ARR6-LUC activity (FIG. 3). The activation of the repressor-type of ARRs is thought to provide a negative feedback loop in controlling the transient induction of cytokinin early response genes and allows resetting and/or fine-tuning of the physiological state of the cells.

Ectopic Expression of the Master Regulator ARR2 Mimics Cytokinin Responses

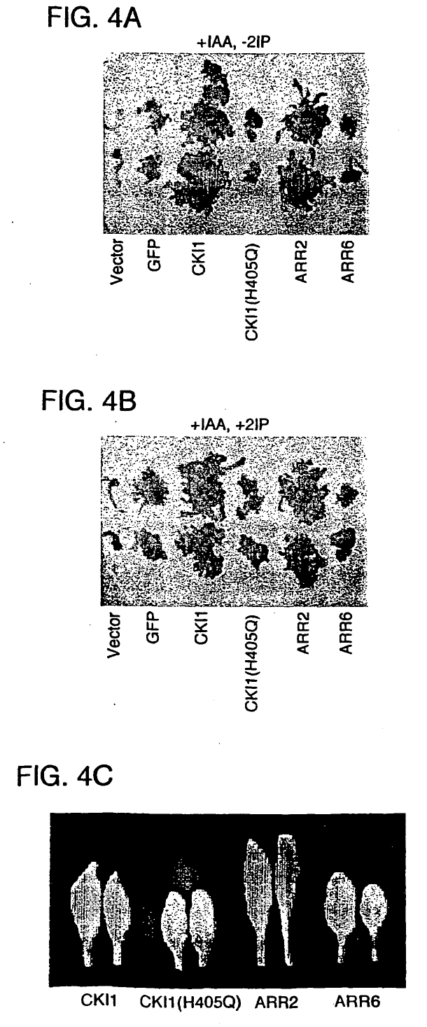

In tissue culture, induction of cell proliferation and subsequent shoot formation require cytokinin. To determine whether the same cytokinin signalling pathway is responsible for transcription regulation, cell proliferation, shoot meristem initiation, and leaf formation, we developed a seedling cytokinin response assay. In FIGS. 4A and 4B, Arabidopsis seedlings were transformed with Agrobacteria carrying GFP, CKI1, CKI1 (H405Q), ARR2, and ARR6 constructs in the mini-binary vector pCB302 (Xiang et al., Plant Mol. Biol. 40: 711-7, 1999). Seedlings transformed with the Agrobacteria carrying the binary vector pCB301 without a Bar selection marker were used as a transformation control (vector). The transformed seedlings were selected with glufosinate ammonium (10 μg/ml) and maintained on medium with auxin (IAA) only (FIG. 4A, +IAA, −2IP) or with auxin and cytokinin (FIG. 4B, +IAA, +2IP) for 14 days. The transformed tissues were mostly derived from the shoot apical meristem. The GFP control showed green callus formation with exogenous IAA but promoted shoot formation with exogenous cytokinin. Both CKI1 and ARR2 promoted extensive cell proliferation and shoot formation even without exogenous cytokinin. The experiments were repeated three times with similar results. Compared with the GFP control, the CKI1(H405Q) mutant and ARR6 slightly inhibited cell proliferation on the same medium without or with exogenous cytokinin (FIGS. 4A and 4B). The results of this cytokinin seedling assay are consistent with those from protoplast transient expression analyses based on transcription. It is therefore clear that the HK activity of CKI1 is required to perceive and/or initiate cytokinin signalling.

To observe the consequences of constitutive cytokinin signalling in planta, the binary vectors expressing CKI1, CKI1(H405Q), ARR2, and ARR6 were introduced into Arabidopsis plants. Ectopic expression of ARR2 in transgenic Arabidopsis plants displayed a spectrum of cytokinin-related phenotypes, including reduced apical dominance, increased leaf number and size, altered leaf shape and color, and delayed leaf senescence (FIG. 4C). Transgenic Arabidopsis plants carrying the CKI1 construct showed similar phenotypes, but not the CKI1(H405Q) and ARR6 transgenic plants serving as controls. Taken together, CKI1 and ARR2 are positive regulators in the cytokinin signal transduction pathway mediating transcription, cell proliferation, and shoot and leaf formation, as well as delaying leaf senescence. ARR2 could be one of the direct downstream targets of CKI1 and CRE1, and serve as a master regulator in diverse cytokinin responses.

Materials and Methods

The above-mentioned results were obtained using the following materials and methods.

Plasmid Constructs

The 2.4 kb Arabidopsis ARR6 promoter was fused to the firefly luciferase gene to create the ARR6-LUC reporter construct. The CKI1 and AHK2 genes were amplified from the Arabidopsis genomic DNA by polymerase chain reaction (PCR). The AHK3, CRE1(CRE1/WOL), AHP1, AHP2, AHP5 and ARR1, ARR 2, ARR4, ARR5, ARR6, ARR7, and ARR10 coding regions were obtained by PCR from an Arabidopsis cDNA library (Sheen, Science 274: 1900-2, 1996). All of the mutants were generated by QuickChange site-directed mutagenesis (Stratagene). The coding regions of all proteins were tagged with either two copies of the haemagglutinin epitope (DHA) or GFP and inserted into a plant expression vector containing the 35SC4PPDK promoter (Sheen, supra) and the nos terminator. All PCR products and the mutations were confirmed by DNA sequencing.

Arabidopsis Protoplast Transient Expression Assay

Arabidopsis Bensheim (BE) protoplasts were isolated and transfected as described with some modifications (Kovtun et al., Proc. Natl. Acad. Sci. USA 97: 2940-5 2000). Typically, 2×10⁴ protoplasts were transfected with 10 μg of plasmid carrying different combinations of a reporter, effectors, and an internal control. For instance, in the experiments shown in FIG. 1A, protoplasts (4×10⁴) were transfected with 2 μg of UBQ10-GUS (internal control) and 20 μg of ARR6-LUC, GH3-LUC, or RD29ALUC plasmid DNA. Transfected protoplasts were incubated at 1×10⁴ per mil with/without 1-100 nM of t-zeatin for 3-6 hours under the light at 23° C. The UBQ10-GUS construct was used as an internal control to normalize the variations of each transfection in cell numbers, transformation efficiency and cell viability. The results are shown as the means of duplicate samples with the standard deviation. All transient experiments were repeated at least three times with similar results. GFP fluorescence was observed by either Nikon TE200 fluorescent microscopy or Leica TCSNT confocal microscopy.

Protein Immunoprecipitation

Transfected protoplasts were incubated with [³⁵S]-methione (200 μCi/ml) for 6 hours. The effector proteins were immunoprecipitated as described (Sheen, supra), analyzed by SDS/PAGE (10 or 12.5%) and visualized by fluorography.

Seedling Cytokinin Response Assay

Four-day-old Arabidopsis BE seedlings were placed on the callus induction medium, containing 0.5 mg/L 2,4-D, 0.05 mg/L BAP, and 0.05 mg/L kinetin, for four days. The seedlings were co-cultivated and transformed with Agrobacterium tumefaciens GV3101carrying pCB302 with either CKI1, CKI1(H405Q), ARR2, ARR6, GFP under control of the 35SC4PPDK promoter, or the pCB301 binary vector (Xiang et al., Plant Mol. Biol. 40:711-7, 1999) alone for three days. Transformed seedlings were then selected on the glufosinate ammonium-containing medium (with either 0.15 mg/L IAA alone or with 0.15 mg/L IAA and 5 mg/L 2IP) and observed up to 4 weeks. The transformed proliferating tissues were mostly derived from the apical shoot meristem of the seedlings.

Transgenic Plant Analysis

The same constructs analyzed in the Arabidopsis protoplast transient expression and seedling assays,including CKI1, CKI1(H405Q), ARR2, and ARR6, were used to generate Arabidopsis transgenic plants using the floral dip method and Bar selection as described (Clough and Bent, Plant J. 16: 735-43, 1998). More than 200 transgenic plants were obtained with each construct. The ARR2 and CKI1 transgenic plants developed branches early, produced more leaves that are bigger and greener but flowered later than the control CKI1(H405Q) and ARR6 transgenic plants. For senescence assay, fully expanded fourth leaves from representative transgenic plants were detached and floated on distilled water in the dark for four days.

Isolation of Sequences Encoding A-type and B-type Response Regulators or Histidine Kinases

The isolation of additional genes encoding A-type and B-type response regulators or histidine kinase genes is accomplished using standard strategies and techniques that are well known in the art.

In one particular example, the B-type response regulator sequence (for example, ARR1, ARR2, or ARR10 sequences), the A-type response regulator sequence, (for example, ARR4, ARR5, ARR6, or ARR7), or the histidine kinase (for example, CRE1 or CKI1), described herein may be used, together with conventional screening methods of nucleic acid hybridization screening, to isolate additional sequences encoding these regulators. Such hybridization techniques and screening procedures are well known to those skilled in the art and are described, for example, in Benton and Davis, Science 196: 180, 1977; Grunstein and Hogness, Proc. Natl. Acad. Sci., USA 72: 3961, 1975; Ausubel et al. Current Protocols in Molecular Biology, Wiley Interscience, New York; Berger and Kimmel, Guide to Molecular Cloning Techniques, 1987, Academic Press, New York.; and Sambrook et al., Molecular Cloning: A Laboratory Manual, Cold Spring Harbor Laboratory Press, New York. In one particular example, all or part of the response regulator gene may be used as a probe,to screen a recombinant plant DNA library for genes having sequence identity or similarity to the response regulator gene. Hybridizing sequences are detected by plaque or colony hybridization according to the methods described below.

Alternatively, using all or a portion of the amino acid sequence of a response regulator, one may readily design oligonucleotide probes, including degenerate oligonucleotide probes (i.e., a mixture of all possible coding sequences for a given amino acid sequence). These oligonucleotides may be based upon the sequence of either DNA strand and any appropriate portion of the response regulator sequence. General methods for designing and preparing such probes are provided, for example, in Ausubel et al., supra; and Berger and Kimmel, supra. These oligonucleotides are useful for response regulator or histidine kinase sequence isolation, either through their use as probes capable of hybridizing to response regulator complementary sequences or as primers for various amplification techniques, for example, polymerase chain reaction (PCR) cloning strategies. If desired, a combination of different oligonucleotide probes may be used for the screening of a recombinant DNA library. The oligonucleotides may be detectably-labeled using methods known in the art and used to probe filter replicas from a recombinant DNA library. Recombinant DNA libraries are prepared according to methods well known in the art, for example, as described in Ausubel et al. (supra), or they may be obtained from commercial sources.

As discussed above, response regulator-specific oligonucleotides may also be used as primers in amplification cloning strategies, for example, using PCR. PCR methods are well known in the art and are described, for example, in PCR Technology, Erlich, ed., Stockton Press, London, 1989; PCR Protocols: A Guide to Methods and Applications, Innis et al., eds., Academic Press, Inc., New York, 1990; and Ausubel et al. (supra). Primers are optionally designed to allow cloning of the amplified product into a suitable vector, for example, by including appropriate restriction sites at the 5′ and 3′ ends of the amplified fragment (as described herein). If desired, response regulator sequences may be isolated using the PCR “RACE” technique, or Rapid Amplification of cDNA Ends (see, e.g., Innis et al. (supra)). By this method, oligonucleotide primers based on the response regulator sequence are oriented in the 3′ and 5′ directions and are used to generate overlapping PCR fragments. These overlapping 3′- and 5′-end RACE products are combined to produce an intact full-length cDNA. This method is described in Innis et al. (supra); and Frohman et al., Proc. Natl. Acad. Sci. USA 85: 8998, 1988.

Confirmation of a sequence's relatedness to the response regulators may be accomplished by a variety of conventional methods including, but not limited to, sequence comparison of the gene and its expressed product to known response regulators for example those disclosed herein. In addition, the activity of the gene product may be evaluated according to any of the techniques described herein.

Once a response regulator gene is identified, it is cloned according to standard methods and used for the construction of plant expression vectors as described below.

Expression Constructs

Those skilled in the field of molecular biology will understand that any of a wide variety of expression systems may be used to express the response regulators disclosed herein. For example, an A-type or B-type response regulator may be expressed for the desired effect in any of a number of plant hosts including, without limitation, algae, tree species, ornamental species, temperate fruit species, tropical fruit species, vegetable species, legume species, crucifer species, monocots, dicots, or in any plant of commercial or agricultural significance. Particular examples of suitable plant hosts include, but are not limited to, Conifers, Petunia, Tomato, Potato, Tobacco, Arabidopsis, Lettuce, Sunflower, Oilseed rape, Flax, Cotton, Sugarbeet, Celery, Soybean, Alfalfa, Medicago, Lotus, Vigna, Cucumber, Carrot, Eggplant, Cauliflower, Horseradish, Morning Glory, Poplar, Walnut, Apple, Grape, Asparagus, Rice, Maize, Millet, Onion, Barley, Orchard grass, Oat, Rye, and Wheat. In addition, as is discussed below, transgenic expression constructs may be expressed in a plant to increase plants yield, shoot formation, leaf growth, photosynthesis, or delay senescence.

Materials for expressing these genes are available from a wide range of sources including the American Type Culture Collection (Rockland, Md.); or from any of a number seed companies, for example, W. Atlee Burpee Seed Co. (Warminster, Pa.), Park Seed Co. (Greenwood, S.C.), Johnny Seed Co. (Albion, Me.), or Northrup King Seeds (Harstville, S.C.). Descriptions and sources of useful host cells are also found in Vasil, Cell Culture and Somatic Cell Genetics of Plants, Vol I, II, III Laboratory Procedures and Their Applications Academic Press, New York, 1984; Dixon, Plant Cell Culture-A Practical Approach, IRL Press, Oxford University, 1985; Green et al., Plant Tissue and Cell Culture, Academic Press, New York, 1987; and Gasser and Fraley, Science 244: 1293, 1989.

The method of transformation or transfection and the choice of vehicle for expression of the A-type or B-type response regulators, or a histidine kinase will depend on the host system selected. Transformation and transfection methods are described, e.g., in Ausubel et al. (supra); Weissbach and Weissbach, Methods for Plant Molecular Biology, Academic Press, 1989; Gelvin et al., Plant Molecular Biology Manual, Kluwer Academic Publishers, 1990; Kindle, Proc. Natl. Acad. Sci., USA 87: 1228, 1990; Potrykus, Annu. Rev. Plant Physiol. Plant Mol. Biology 42: 205, 1991; and BioRad (Hercules, Calif.) Technical Bulletin #1687 (Biolistic Particle Delivery Systems). Expression vehicles may be chosen from those provided, e.g., in Cloning Vectors: A Laboratory Manual (Pouwels et al., 1985, Supp. 1987); Gasser and Fraley (supra); Clontech Molecular Biology Catalog (Catalog 1992/93 Tools for the Molecular Biologist, Palo Alto, Calif.); and the references cited above. Other expression constructs are described by Fraley et al. (U.S. Pat. No. 5,352,605).

Most preferably, an A-type or B-type response regulator or a histidine kinase is produced by a stably-transfected plant cell line, a transiently-transfected plant cell line, or by a transgenic plant. A number of vectors suitable for stable transfection of plant cells or for the establishment of transgenic plants are available to the public; such vectors are described in Pouwels et al. (supra), Weissbach and Weissbach (supra), and Gelvin et al. (supra). Methods for constructing such cell lines are described in, e.g., Weissbach and Weissbach (supra), and Gelvin et al. (supra). Typically, plant expression vectors include (1) a cloned plant gene under the transcriptional control of 5′ and 3′ regulatory sequences and (2) a dominant selectable marker. Such plant expression vectors may also contain, if desired, a promoter regulatory region (for example, one conferring inducible or constitutive, pathogen- or wound-induced, environmentally- or developmentally-regulated, or cell- or tissue-specific expression), a transcription initiation start site, a ribosome binding site, an RNA processing signal, a transcription termination site, and/or a polyadenylation signal.

Once the desired nucleic acid sequence encoding an A-type or B-type response regulators or a histidine kinase is obtained as described above, it may be manipulated in a variety of ways known in the art. For example, where the sequence involves non-coding flanking regions, the flanking regions may be subjected to mutagenesis.

In general, the constructs will involve regulatory regions functional in plants which provide for modified production of the regulator protein or regulator transcript (such as an antisense transcript) as discussed herein. The open reading frame coding for the regulator protein or functional fragment thereof will be joined at its 5′ end to a transcription initiation regulatory region such as the sequence naturally found in the 5′ upstream region of the A-type or B-type response regulator, or a histidine kinase. Numerous other transcription initiation regions are available which provide for constitutive or inducible regulation.

For applications where developmental, cell, tissue, hormonal, or environmental expression is desired, appropriate 5′ upstream non-coding regions are obtained from other genes, for example, from genes regulated during meristem development, seed development, embryo development, or leaf development.

Regulatory transcript termination regions may also be provided in DNA constructs of this invention as well. Transcript termination regions may be provided by the DNA sequence encoding the A-type or B-type response regulator or histidine kinase, or any convenient transcription termination region derived from a different gene source. The transcript termination region will contain preferably at least 1-3 kb of sequence 3′ to the structural gene from which the termination region is derived. Plant expression constructs having, for example, A-type or B-type response regulator as the DNA sequence of interest for expression may be employed with a wide variety of plant life. Such genetically-engineered plants are useful for a variety of industrial and agricultural applications as discussed herein. Importantly, this invention is applicable to dicotyledons and monocotyledons, and will be readily applicable to any new or improved transformation or regeneration method.

An example of a useful plant promoter according to the invention is a caulimovirus promoter, for example, a cauliflower mosaic virus (CaMV) promoter. These promoters confer high levels of expression in most plant tissues, and the activity of these promoters is not dependent on virally encoded proteins. CaMV is a source for both the 35S and 19S promoters. In most tissues of transgenic plants, the CaMV 35S promoter is a strong promoter (see, e.g., Odell et al., Nature 313: 810, 1985). The CaMV promoter is also highly active in nmonocots (see, e.g., Dekeyser et al., Plant Cell 2: 591, 1990; Terada and Shimamoto, Mol Gen. Genet. 220: 389,1990). Moreover, activity of this promoter can be further increased (i.e., between 2-10 fold) by duplication of the CaMV 35S promoter (see e.g., Kay et al., Science 236: 1299, 1987; Ow et al., Proc. Natl. Acad Sci., U.S.A. 84: 4870, 1987; and Fang et al., Plant Cell 1: 141, 1989). In addition, the a minimal 35S promoter may also be used as is described herein.

Other useful plant promoters include, without limitation, the nopaline synthase promoter (An et al., Plant Physiol. 88: 547, 1988) and the octopine synthase promoter (Fromm et al., Plant Cell 1: 977, 1989).

For certain applications, it may be desirable to produce the regulator in an appropriate tissue, at an appropriate level, or at an appropriate developmental time. For this purpose, there are an assortment of gene promoters, each with its own distinct characteristics embodied in its regulatory sequences, shown to be regulated in response to the environment, hormones, and/or developmental cues. These include gene promoters that are responsible for heat-regulated gene expression (see, e.g., Callis et al., Plant Physiol. 88: 965, 1988; Takahashi and Komeda, Mol. Gen. Genet. 219: 365, 1989; and Takahashi et al., Plant J. 2: 751, 1992), light-regulated gene expression (e.g., the Arabidopisis Cab2 photosynthetic, leaf specific promoter described by Mitra et al., Plant Mol. Biol. 12: 169-179, 1989; the pea rbcS-3A described by Kuhlemeier et al., Plant Cell 1: 471, 1989; the maize rbcS promoter described by Schäffner and Sheen, Plant Cell 3: 997, 1991; or the cholorphyll a/b-binding protein gene found in pea described by Simpson et al., EMBO J. 4: 2723, 1985), hormone-regulated gene expression (for example, the abscisic acid (ABA) responsive sequences from the Em gene of wheat described by Marcotte et al., Plant Cell 1: 969, 1989; the ABA-inducible HVA1 and HVA22, and rd29A promoters described for barley and Arabidopsis by Straub et al., Plant Cell 6: 617, 1994, Shen et al., Plant Cell 7: 295, 1995), wound-induced gene expression (for example, of wunI described by Siebertz et al., Plant Cell 1: 961, 1989), and organ-specific gene expression (for example, of the tuber-specific storage protein gene described by Roshal et al., EMBO J. 6: 1155,1987; the 23-kDa zein gene from maize described by Schernthaner et al., EMBO J. 7: 1249, 1988; or the French bean β-phaseolin gene described by Bustos et al., Plant Cell 1: 839, 1989; the vegetative storage protein promoter (soybean vspB) described by Sadka et al. (Plant Cell 6: 737-749, 1994)), cycling promoters (e.g., the Arabidopsis cdc2a promoter described by Hemerly et al., Proc. Natl. Acad. Sci. USA 89: 3295-3299, 1992), senescence-specific promoters (e.g., the Arabidopsis SAG12 promoter described by Gan et al, Science: 270, 1986-1988, 1995), seed-specific promoters (for example, endosperm-specific or embryo-specific promoters), or pathogen-inducible promoters (for example, PR-1 or β-1,3 glucanase promoters).

Plant expression vectors may also optionally include RNA processing signals, e.g, introns, which have been shown to be important for efficient RNA synthesis and accumulation (Callis et al., Genes and Dev. 1: 1183, 1987). The location of the RNA splice sequences can dramatically influence the level of transgene expression in plants. In view of this fact, an intron may be positioned upstream or downstream of the regulator encoding sequence in the transgene to modulate levels of gene expression.

In addition to the aforementioned 5′ regulatory control sequences, the expression vectors may also include regulatory control regions which are generally present in the 3′ regions of plant genes (Thornburg et al., Proc. Natl. Acad. Sci. U.S.A. 84: 744, 1987; An et al., Plant Cell 1: 115, 1989). For example, the 3′ terminator region may be included in the expression vector to increase stability of the mRNA. One such terminator region may be derived from the PI-II terminator region of potato. In addition, other commonly used terminators are derived from the octopine or nopaline synthase signals.

The plant expression vector also typically contains a dominant selectable marker gene used to identify those cells that have become transformed. Useful selectable genes for plant systems include genes encoding antibiotic resistance, for example, those encoding resistance to hygromycin, kanamycin, bleomycin, G418, streptomycin, or spectinomycin. Genes required for photosynthesis may also be used as selectable markers in photosynthetic-deficient strains. Finally, genes encoding herbicide resistance may be used as selectable markers; useful herbicide resistance genes include the bar gene encoding the enzyme phosphinothricin acetyltransferase and conferring resistance to the broad spectrum herbicide Basta® (Hoechst AG, Frankfurt, Germany).

Efficient use of selectable markers is facilitated by a determination of the susceptibility of a plant cell to a particular selectable agent and a determination of the concentration of this agent which effectively kills most, if not all, of the untransformed cells. Some useful concentrations of antibiotics for tobacco transformation include, e.g., 75-100 μg/mL (kanamycin), 20-50 μg/mL (hygromycin), or 5-10 μg/mL (bleomycin). A useful strategy for selection of transformants for herbicide resistance is described, e.g., by Vasil et al., supra.

It should be readily apparent to one skilled in the art of molecular biology, especially in the field of plant molecular biology, that the level of gene expression is dependent, not only on the combination of promoters, RNA processing signals, and terminator elements, but also on how these elements are used to increase the levels of selectable marker gene expression.

Plant Transformation

Upon construction of the plant expression vector, several standard methods are available for introduction of the vector into a plant host, thereby generating a transgenic plant. These methods include (1) Agrobacterium-mediated transformation (A. tumefaciens or A. rhizogenes) (see, e.g., Lichtenstein and Fuller, In: Genetic Engineering, Vol 6, Rigby, ed., London, Academic Press, 1987; and Lichtenstein and Draper, In: DNA Cloning, Vol II, Glover, ed., Oxford, IRI Press, 1985), (2) the particle delivery system (see, e.g., Gordon-Kamin et al., Plant Cell 2: 603, 1990); or BioRad Technical Bulletin #1687, supra), (3) microinjection protocols (see, e.g., Green et al., supra), (4) polyethylene glycol (PEG) procedures (see, e.g., Draper et al., Plant Cell Physiol. 23: 451, 1982; or e.g., Zhang and Wu, Theor. Appl. Genet. 76: 835, 1988), (5) liposome-mediated DNA uptake (see, e.g., Freeman et al., Plant Cell Physiol. 25: 1353, 1984), (6) electroporation protocols (see, e.g., Gelvin et al., supra; Dekeyser et al., supra; Fromm et al., Nature 319: 791, 1986; Sheen, Plant Cell 2: 1027, 1990; or Jang and Sheen, Plant Cell 6: 1665, 1994), (7) the vortexing method (see, e.g., Kindle, supra), and floral dip method (see, e.g., Clough and Bent, Plant J. 16, 735-43. (1998)). The method of transformation is not critical to the invention. Any method which provides for efficient transformation may be employed. As newer methods are available to transform crops or other host cells, they may be directly applied.

The following is an example outlining one particular technique, an Agrobacterium-mediated plant transformation. By this technique, the general process for manipulating genes to be transferred into the genome of plant cells is carried out in two phases. First, cloning and DNA modification steps are carried out in E. coli, and the plasmid containing the gene construct of interest is transferred by conjugation or electroporation into Agrobacterium. Second, the resulting Agrobacterium strain is used to transform plant cells. Thus, for the generalized plant expression vector, the plasmid contains an origin of replication that allows it to replicate in Agrobacterium and a high copy number origin of replication functional in E. coli. This permits facile production and testing of transgenes in E. coli prior to transfer to Agrobacterium for subsequent introduction into plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer unctions of Agrobacterium, delimit the DNA region that will be transferred to the plant.

In another example, plant cells may be transformed by shooting into the cell tungsten microprojectiles on which cloned DNA is precipitated. In the Biolistic Apparatus (Bio-Rad) used for the shooting, a gunpowder charge (22 caliber Power Piston Tool Charge) or an air-driven blast drives a plastic macroprojectile through a gun barrel. An aliquot of a suspension of tungsten particles on which DNA has been precipitated is placed on the front of the plastic macroprojectile. The latter is fired at an acrylic stopping plate that has a hole through it that is too small for the macroprojectile to pass through. As a result, the plastic macroprojectile smashes against the stopping plate, and the tungsten microprojectiles continue toward their target through the hole in the plate. For the instant invention the target can be any plant cell, tissue, seed, or embryo. The DNA introduced into the cell on the microprojectiles becomes integrated into either the nucleus or the chloroplast.

In general, transfer and expression of transgenes in plant cells are now routine practices to those skilled in the art, and have become major tools to carry out gene expression studies in plants and to produce improved plant varieties of agricultural or commercial interest.

Transgenic Plant Regeneration

Whole plants can be regenerated, for example, from single cells, callus tissue, or leaf discs transformed with a plant expression vector according to standard plant tissue culture techniques. It is well known in the art that various cells, tissues, and organs from almost any plant can be successfully cultured to regenerate an entire plant; such techniques are described, e.g., in Vasil, supra; Green et al., supra; Weissbach and Weissbach, supra; and Gelvin et al., supra.

In one particular example, a cloned B-type response regulator or histidine kinase construct under the control of the nos promoter and the nopaline synthase terminator and carrying a selectable marker (for example, kanamycin resistance) is transformed into Agrobacterium. Transformation of leaf discs (for example, of tobacco or potato leaf discs), with vector-containing Agrobacterium is carried out as described by Horsch et al. (Science 227: 1229, 1985). Putative transformants are selected after a few weeks (for example, 3 to 5 weeks) on plant tissue culture media containing kanamycin (e.g. 100 μg/mL). Kanamycin-resistant shoots are then placed on plant tissue culture media without hormones for root initiation. Kanamycin-resistant plants are then selected for greenhouse growth. If desired, seeds from self-fertilized transgenic plants can then be sowed in a soil-less medium and grown in a greenhouse. Kanamycin-resistant progeny are selected by sowing surfaced sterilized seeds on hormone-free kanamycin-containing media. Analysis for the integration of the transgene is accomplished by standard techniques (see, for example, Ausubel et al., supra; Gelvin et al., supra).

Transgenic plants expressing the selectable marker are then screened for transmission of the transgene DNA by standard immunoblot and DNA detection techniques. Each positive transgenic plant and its transgenic progeny are unique in comparison to other transgenic plants established with the same transgene. Integration of the transgene DNA into the plant genomic DNA is in most cases random, and the site of integration can profoundly affect the levels and the tissue and developmental patterns of transgene expression. Consequently, a number of transgenic lines are usually screened for each transgene to identify and select plants with the most appropriate expression profiles.

Transgenic lines are evaluated for levels of transgene expression. Expression at the RNA level is determined initially to identify and quantitate expression-positive plants. Standard techniques for RNA analysis are employed and include PCR amplification assays using oligonucleotide primers designed to amplify only transgene RNA templates and solution hybridization assays using transgene-specific probes (see, e.g., Ausubel et al., supra). The RNA-positive plants are then analyzed for protein expression by Western immunoblot analysis using specific antibodies (see, e.g., Ausubel et al., supra). In addition, in situ hybridization and immunocytochemistry according to standard protocols can be done using transgene-specific nucleotide probes and antibodies, respectively, to localize sites of expression within transgenic tissue.

Increasing Plant Yield

To test the ability of the genes described herein to improve crop yield or productivity, seeds of transgenic plants expressing a B-type response regulator or histidine kinase are planted in test plots, and their agronomic performance is compared to standard plants using techniques familiar to those of skill in the art. Optionally included in this comparison are plants of similar genetic background without the transgene. A yield benefit is observed and plants exhibiting the increased yield are advanced for commercialization.

In addition, transgenic plants expressing a B-type response regulator or histidine kinase are field tested for agronomic performance under conditions, including, but not limited to, increased leaf production. When compared to nontransgenic plants, transgenic plants expressing the B-type response regulator exhibit higher yield than their non-transgenic counterparts.

Increasing Plant Shoot Formation

To test the ability of the genes described herein to increase shoot formation, seedlings are transformed with a transgene expressing a B-type response regulator or histidine kinase and shoot formation is measured, for example, by evaluating the development of green callus formation, using techniques familiar to those of skill in the art. Optionally included in this comparison are plants of similar genetic background without the transgene. Plants exhibiting increased shoot formation relative to control plants are useful in the invention.

Delaying Plant Senescence

To test the ability of the genes described herein to delay senescence, fully expanded fourth leaves of plants expressing a recombinant B-type response regulator or histidine kinase are detached and floated on distilled water in the dark for four days, and their leaf size and quality of green color are compared using techniques familiar to those of skill in the art. Optionally included in this comparison are plants of similar genetic background without the transgene.

Enhancing Plant Photosynthesis

To test the ability of the genes and domains described herein to enhance photosynthesis, seedlings are transformed with a transgene expressing a recombinant B-type response regulator or histidine kinase and photosynthesis measured using techniques familiar to those of skill in the art. Optionally included in this comparison are plants of similar genetic background without the transgene.

Increasing Plant Leaf Growth

To test the ability of the genes described herein to increase leaf growth, seedlings are transformed with a transgene expressing a recombinant B-type response regulator and leaf growth measured, for example, by measuring leaf number and leaf area using techniques familiar to those of skill in the art. Optionally included in this comparison are plants of similar genetic background without the transgene.

Silencing A-type Response Regulator Gene Expression

Plants having decreased expression of an A-type response regulator are useful, for example, for increasing plant productivity, delaying senescence, increasing photosynthesis, increased leaf growth, and increased productivity. Plants having decreased expression of such A-type response regulators are generated according to standard gene silencing methods including, without limitation, co-suppression and antisense methodologies, expression of dominant negative gene products, and creation of plants having mutated genes encoding such regulators.

Co-Suppression

One preferred method of silencing gene expression is co-suppression (also referred to as sense suppression). This technique, which involves introduction of a nucleic acid configured in the sense orientation, has been shown to effectively block the transcription of target genes (see for example, Napoli et al., Plant Cell, 2:279-289, 1990 and Jorgensen et al., U.S. Pat. No. 5,034,323).

Generally, sense suppression involves transcription of the introduced sequence such as a gene encoding an A-type response regulator. However, co-suppression may also occur where the introduced sequence contains no coding sequence per se, but only intron or untranslated sequences homologous to sequences present in the primary transcript of the endogenous gene to be repressed. The introduced sequence generally will be substantially identical to the endogenous gene targeted for repression. Such identity is typically greater than about 50%, but higher identities (for example, 80% or even 95%) are preferred because they result in more effective repression. The effect of co-suppression may also be applied to other proteins within a similar family of genes exhibiting homology or substantial homology. Segments from a gene from one plant can be used directly, for example, to inhibit expression of homologous genes in different plant species.

In sense suppression, the introduced sequence, requiring less than absolute identity, need not be full length, relative to either the primary transcription product or to fully processed mRNA. A higher identity in a shorter than full length sequence compensates for a longer sequence with less identity. Furthermore, the introduced sequence need not have the same intron or exon pattern, and identity of non-coding segments may be equally effective. Sequences of at least 50 base pairs are preferred, with introduced sequences of greater length being more preferred (see, for example, those methods described by Jorgensen et al., supra).

Antisense Suppression

In antisense technology, a nucleic acid segment from the desired plant gene is cloned and operably linked to an expression control region such that the antisense strand of RNA is synthesized. The construct is then transformed into plants and the antisense strand of RNA is produced. In plant cells, it has been shown that antisense RNA inhibits gene expression.

The nucleic acid segment to be introduced in antisense suppression is generally substantially identical to at least a portion of the endogenous gene or genes to be repressed, but need not be identical. The vectors of the present invention therefore can be designed such that the inhibitory effect applies to other proteins within a family of genes exhibiting homology or substantial homology to the target gene. Segments from a gene from one plant can be used, for example, directly to inhibit expression of homologous genes in different plant species.

The introduced sequence also need not be full length relative to either the primary transcription product or to fully processed mRNA. Generally, higher homology can be used to compensate for the use of a shorter sequence. Moreover, the introduced sequence need not have the same intron or exon pattern, and homology of non-coding segments will be equally effective. In general, such an antisense sequence will usually be at least 15 base pairs, preferably about 15-200 base pairs, and more preferably 200-2,000 base pairs in length or greater. The antisense sequence may be complementary to all or a portion of the gene to be suppressed and as appreciated by those skilled in the art, the particular site or sites to which the antisense sequence binds as well as the length of the antisense sequence will vary, depending upon the degree of inhibition desired and the uniqueness of the antisense sequence. A transcriptional construct expressing a plant A-type response regulator (e.g., those described herein) antisense nucleotide sequence includes, in the direction of transcription, a promoter, the sequence coding for the antisense RNA on the sense strand, and a transcriptional termination region. Antisense sequences may be constructed and expressed as described, for example, in van der Krol et al. (Gene 72: 45-50, 1988); Rodermiel et al. (Cell 55: 673-681, 1988); Mol et al. (FEBS Lett. 268: 427-430, 1990); Weigel and Nilsson (Nature 377: 495-500, 1995); Cheung et al., (Cell 82: 383-393, 1995); and Shewmaker et al. (U.S. Pat. No. 5,107,065).

In one working example, antisense expression of the gene encoding A-type response regulators of Arabidopsis or an A-type response regulator homolog is used to increase plant yield. In one particular approach, a plant expression vector is constructed that contains the cDNA sequence of A-type response regulator homolog in antisense orientation that is constitutively expressed under the control of the rice actin promoter described by Wu et al. (WO 91/09948). This expression vector is then used to transform rice plants according to conventional methods, for example, using the methods described in Hiei et al. (Plant J. 6:271-282, 1994).

Dominant Negatives

Transgenic plants expressing a transgene encoding a dominant negative gene product of an A-type response regulator are assayed in artificial environments or in the field to demonstrate that the transgene increases yield, delays senescence, increases photosynthesis, or increases leaf formation on the plant expressing the gene. Dominant negative transgenes are constructed according to methods known in the art. Typically, a dominant negative gene encodes a mutant A-type response regulator which, when overexpressed, disrupts the activity of the wild type polypeptide.

Mutants

Plants having decreased expression of an A-type response regulator are also generated using standard mutagenesis methodologies. Such mutagenesis methods include, without limitation, treatment of seeds with ethyl methylsulfate, ethylmethylsulfonate (EMS), or fast neutron irradiation, as well as T-DNA insertion methodologies. Expression of an A-type response regulator and increased yield phenotypes in mutated and non-mutated lines are evaluated according to standard procedures (for example, those described herein). Mutated plants having decreased expression of a gene encoding a A-type response regulator exhibit increased yield relative to their non-mutated counterparts.

Use

The invention described herein is useful for a variety of agricultural and commercial purposes including, but not limited to, improving and enhancing photosynthesis, promoting cell proliferation, shoot meristem formation, promoting leaf development, increasing crop yields, improving crop and ornamental quality, and reducing agricultural production costs. In particular, ectopic expression of a B-type response regulator (such as ARR2 or an ARR2 homolog) in a plant cell provides a method for increasing plant productivity, photosynthesis, and delaying senescence. The invention is especially useful for crop plants such as tomato, potato, cotton, pepper, maize, wheat, rice, and legumes such as soybean.

All publications and patent applications mentioned in this specification are herein incorporated by reference to the same extent as if each independent publication or patent application was specifically and individually indicated to be incorporated by reference.

What is claimed is:

1. A method for increasing yield in a plant, said method comprising the steps of:
(a) introducing into plant cells a transgene comprising DNA encoding a B-type response regulator operably linked to a promoter functional in plant cells to yield transformed plant cells; and
(b) regenerating a plant from said transformed cells, wherein said B-type response regulator is expressed in the cells of said transgenic plant, thereby increasing yield in said plant.

2. The method of claim 1, wherein said B-type response regulator is a crucifer B-type response regulator.

3. The method of claim 2, wherein said crucifer B-type response regulator is selected from the group consisting of ARR1, ARR2, and ARR10.

4. The method of claim 1, wherein said DNA encoding said B-type response regulator is constitutively expressed, inducibly expressed, expressed in a cell-specific, tissue-specific, or organ-specific manner, or expressed under cycling conditions.

5. A method for increasing yield in a plant, said method comprising the steps of:
(a) introducing into plant cells a transgene operably linked to a promoter functional in plant cells to yield transformed plant cells; and
(b) regenerating a plant from said transformed cells, wherein expression of said transgene reduces expression of an A-type response regulator in the cells of said plant, thereby increasing yield in said plant.

6. The method of claim 5, wherein said A-type response regulator is a crucifer A-type response regulator.

7. The method of claim 6, wherein said crucifer A-type response regulator is selected from the group consisting of ARR4, ARR5, ARR6, and ARR7.

8. The method of claim 5, wherein said transgene expresses antisense A-type response regulator RNA.

9. The method of claim 5, wherein said transgene expresses a dominant negative A-type response regulator.

10. The method of claim 5, wherein said transgene co-suppresses expression of A-type response regulator.

11. The method of claim 5, wherein said DNA encoding said transgene is constitutively expressed, inducibly expressed, expressed in a cell-specific, tissue-specific, or organ-specific manner, or expressed under cycling conditions.

12. A method for increasing shoot formation in a plant, said method comprising the steps of:
(a) introducing into plant cells a transgene comprising DNA encoding a B-type response regulator operably linked to a promoter functional in plant cells to yield transformed plant cells; and
(b) regenerating a plant from said transformed cells, wherein said B-type response regulator is expressed in the cells of said plant, thereby increasing shoot formation in said plant.

13. The method of claim 12, wherein said B-type response regulator is a crucifer B-type response regulator.

14. The method of claim 13, wherein said crucifer B-type response regulator is selected from the group consisting of ARR1, ARR2, and ARR10.

15. The method of claim 12, wherein said DNA encoding said B-type response regulator is constitutively expressed, inducibly expressed, expressed in a cell-specific, tissue-specific, or organ-specific manner, or expressed under cycling conditions.

16. A method for increasing shoot formation in a plant, said method comprising the steps of:
(a) introducing into plant cells a transgene operably linked to a promoter functional in plant cells to yield transformed plant cells; and
(b) regenerating a plant from said transformed cells, wherein expression of said transgene reduces expression of an A-type response regulator in the cells of said plant, thereby increasing shoot formation in said plant.

17. The method of claim 16, wherein said A-type response regulator is a crucifer A-type response regulator.

18. The method of claim 17, wherein said crucifer A-type response regulator is selected from the group consisting of ARR4, ARR5, ARR6, and ARR7.

19. The method of claim 16, wherein said transgene expresses antisense A-type response regulator RNA.

20. The method of claim 16, wherein said transgene expresses a dominant negative A-type response regulator.

21. The method of claim 16, wherein said transgene co-suppresses expression of A-type response regulator.

22. The method of claim 16, wherein said transgene is constitutively expressed, inducibly expressed, expressed in a cell-specific, tissue-specific, or organ-specific manner, or expressed under cycling conditions.

23. A method for delaying senescence in a plant, said method comprising the steps of:
(a) introducing into plant cells a transgene comprising DNA encoding a B-type response regulator operably linked to a promoter functional in plant cells to yield transformed plant cells; and
(b) regenerating a plant from said transformed cells, wherein said B-type response regulator is expressed in the cells of said plant, thereby delaying senesence in said plant.

24. The method of claim 23, wherein said B-type response regulator is a crucifer B-type response regulator.

25. The method of claim 24, wherein said crucifer B-type response regulator is selected from the group consisting of ARR1, ARR2, and ARR10.

26. The method of claim 23, wherein said DNA encoding said B-type response regulator is constitutively expressed, inducibly expressed, expressed in a cell-specific, tissue-specific, or organ-specific manner, or expressed under cycling conditions.

27. A method for delaying senescence in a plant, said method comprising the steps of:
(a) introducing into plant cells a transgene operably linked to a promoter functional in plant cells to yield transformed plant cells; and
(b) regenerating a plant from said transformed cells, wherein expression of said transgene reduces expression of an A-type response regulator in the cells of said plant, thereby delaying senescence in said plant.

28. The method of claim 27, wherein said A-type response regulator is a crucifer A-type response regulator.

29. The method of claim 28, wherein said crucifer A-type response regulator is selected from the group consisting of ARR4, ARR5, ARR6, and ARR7.

30. The method of claim 27, wherein said transgene expresses antisense A-type response regulator RNA.

31. The method of claim 27, wherein said transgene expresses a dominant negative A-type response regulator.

32. The method of claim 27, wherein said transgene co-suppresses expression of A-type response regulator.

33. The method of claim 27, wherein said transgene is constitutively expressed, inducibly expressed, expressed in a cell-specific, tissue-specific, or organ-specific manner, or expressed under cycling conditions.

34. A method for increasing yield in a plant, said method comprising the steps of:
(a) introducing into plant cells a transgene comprising DNA encoding a histidine kinase operably linked to a promoter functional in plant cells to yield transformed plant cells; and
(b) regenerating a plant from said transformed cells, wherein histidine kinase is expressed in the cells of said plant, thereby increasing yield in said plant.

35. The method of claim 34, wherein said histidine kinase is a crucifer histidine kinase.

36. The method of claim 35, wherein said crucifer histidine kinase is CKI1 or CRE1.

37. The method of claim 34, wherein said DNA encoding said histidine kinase is constitutively expressed, inducibly expressed, expressed in a cell-specific, tissue-specific, or organ-specific manner, or expressed under cycling conditions.

38. A method for increasing shoot formation in a plant, said method comprising the steps of:
(a) introducing into plant cells a transgene comprising DNA encoding a histidine kinase operably linked to a promoter functional in plant cells to yield transformed plant cells; and
(b) regenerating a plant from said transformed cells, wherein histidine kinase is expressed in the cells of said plant, thereby increasing shoot formation in said plant.

39. The method of claim 38, wherein said histidine kinase is a crucifer histidine kinase.

40. The method of claim 39, wherein said crucifer histidine kinase is CKI1 or CRE1.

41. The method of claim 38, wherein said DNA encoding said histidine kinase is constitutively expressed, inducibly expressed, expressed in a cell-specific, tissue-specific, or organ-specific manner, or expressed under cycling conditions.

42. A method for delaying senescence in a plant, said method comprising the steps of:
(a) introducing into plant cells a transgene comprising DNA encoding a histidine kinase operably linked to a promoter functional in plant cells to yield transformed plant cells; and
(b) regenerating a plant from said transformed cells, wherein histidine kinase is expressed in the cells of said plant, thereby delaying senescence in said plant.

43. The method of claim 42, wherein said histidine kinase is a crucifer histidine kinase.

44. The method of claim 43, wherein said crucifer histidine kinase is CKI1 or CRE1.

45. The method of claim 42, wherein said DNA encoding said histidine kinase is constitutively expressed, inducibly expressed, expressed in a cell-specific, tissue-specific, or organ-specific manner, or expressed under cycling conditions.

46. A plant or plant component comprising at least one transgene encoding (i) an A-type response regulator polypeptide, (ii) an antisense A-type response regulator RNA, (iii) a dominant negative A-type response regulator polypeptide, (iv) a B-type response regulator polypeptide, (v) an antisense B-type response regulator RNA, (vi) a dominant negative B-type response regulator, (vii) an antisense HK RNA, (viii) a dominant negative HK polypeptide, or any combination of (i)-(viii).

47. The plant or plant component of claim 48, further comprising a transgene encoding a histidine kinase polypeptide.

48. A plant or plant component comprising:
(a) a first transgene encoding (i) an A-type response regulator polypeptide, (ii) an antisense A-type response regulator RNA, or (iii) a dominant negative A-type response regulator polypeptide or combination thereof,
(b) a second transgene encoding (iv) a B-type response regulator polypeptide, (v) an antisense B-type response regulator RNA, (vi) a dominant negative B-type response regulator or combination thereof, and
(c) a third transgene encoding (vii) a HK polypeptide; (viii) an antisense HK RNA, (ix) a dominant negative HK polypeptide or combination thereof.

49. The plant or plant component of claim 48, wherein said plant or plant component is selected from the group consisting of wheat, rice, maize, barley, potato, soybean, tomato, oats, cotton, and sunflower.

50. The plant or plant component of claim 46, wherein said plant or plant component is selected from the group consisting of wheat, rice, maize, barley, potato, soybean, tomato, oats, cotton, and sunflower.

51. The plant or plant component of claim 47, wherein said plant or plant component is selected from the group consisting of wheat, rice, maize, barley, potato, soybean, tomato, oats, cotton, and sunflower.
